(19) United States
(12) Patent Application Publication
(10) Pub. No.: US 2021/0000355 A1
(43) Pub. Date: Jan. 7, 2021

(54) STRESS EVALUATION DEVICE, STRESS EVALUATION METHOD, AND NON-TRANSITORY COMPUTER-READABLE MEDIUM

(71) Applicant: Panasonic Intellectual Property Management Co., Ltd., Osaka (JP)

(72) Inventors: TAKEHIRO ZUKAWA, Shiga (JP); YUKIHIRO MORITA, Osaka (JP)

(21) Appl. No.: 17/029,128

(22) Filed: Sep. 23, 2020

Related U.S. Application Data
(63) Continuation of application No. PCT/JP2019/016437, filed on Apr. 17, 2019.

(30) Foreign Application Priority Data
May 30, 2018 (JP) 2018-103049
May 30, 2018 (JP) 2018-103066
Mar. 29, 2019 (JP) 2019-067219

(51) Int. Cl. A61B 5/024; A61B 5/00; A61B 5/01; A61B 5/053

(52) U.S. Cl. CPC A61B 5/02405; A61B 5/053; A61B 5/01; A61B 5/7296

(57) ABSTRACT

A stress evaluation device includes a first sensor that measures a heart rate and a heart rate variability of a measurement subject; a calculator that calculates (i) an amount of change in heart rate and (ii) an amount of change in heart rate variability; and a determiner that determines a factor for stress of the measurement subject in accordance with the (i) and the (ii) and that outputs a determination result. The amount of change in heart rate is an amount of change from a reference value to the measured heart rate. The amount of change in heart rate variability is an amount of change from a reference value to the measured heart rate variability. The determiner makes (I) a comparison between the (i) and a first threshold, and (II) a comparison between the (ii) and a second threshold to determine the factor for the stress.

### BACKGROUND

### 1. Technical Field

The present disclosure relates to a stress evaluation device, a stress evaluation method, and a non-transitory computer-readable medium that determine a factor for stress of a measurement subject.

### 2. Description of the Related Art

The recent development in wearable devices has spread the use of biological index measurement devices capable of measuring a biological index in daily life. For example, in stress evaluation devices, attempts have been made in which a motion of a measurement subject is detected by an acceleration sensor mounted in the device to measure at-rest stress.

For example, Japanese Unexamined Patent Application Publication No. 2009-148372 (hereinafter Patent Document 1) discloses a system capable of calculating an activity intensity or the like of a measurement subject on the basis of a detection value of an acceleration sensor and determining a stress state of the measurement subject on the basis of the activity intensity and a biological index, such as heart rate, heartbeat waveform, blood pressure, blood oxygen saturation, body temperature, or perspiration level.

Japanese Unexamined Patent Application Publication No. 2001-344352 (hereinafter Patent Document 2) discloses a life support device and a life support method that analyze and determine a stress state of a measurement subject together with a surrounding situation on the basis of a biological index and action information of the measurement subject, thereby providing the measurement subject with a stress relief method or the like.

### SUMMARY

One non-limiting and exemplary embodiment provides a stress evaluation device, a stress evaluation method, and a non-transitory computer-readable medium that are capable of determining a factor for stress of a measurement subject.

In one general aspect, the techniques disclosed here feature a stress evaluation device including a first sensor that measures a heart rate and a heart rate variability of a measurement subject; a calculator that calculates (i) an amount of change in heart rate and (ii) an amount of change in heart rate variability; and a determiner that determines a factor for stress of the measurement subject in accordance with (i) the amount of change in heart rate and (ii) the amount of change in heart rate variability and that outputs information based on a determination result. The amount of change in heart rate is an amount of change from a reference value that is an at-rest heart rate of the measurement subject to the heart rate measured by the first sensor. The amount of change in heart rate variability is an amount of change from a reference value that is an at-rest heart rate variability of the measurement subject to the heart rate variability measured by the first sensor. The determiner makes (I) a comparison between relative magnitudes of the amount of change in heart rate and a first threshold, and (II) a comparison between relative magnitudes of the amount of change in heart rate variability and a second threshold to determine the factor for the stress.

It should be noted that general or specific embodiments may be implemented as a system, a device, a method, an integrated circuit, a computer program, a computer-readable recording medium such as a compact disc-read only memory (CD-ROM), or any selective combination thereof.

A stress evaluation device, a stress evaluation method, and a non-transitory computer-readable medium according to one embodiment of the present disclosure are capable of evaluating a factor for stress of a measurement subject.

Additional benefits and advantages of the disclosed embodiments will become apparent from the specification and drawings. The benefits and/or advantages may be individually acquired by the various embodiments and features of the specification and drawings, which need not all be provided in order to obtain one or more of such benefits and/or advantages.

### BRIEF DESCRIPTION OF THE DRAWINGS

FIG. 1 is a graph that plots the amounts of change in biological indices of twenty subjects for individual factors for stress;

FIG. 2 is a graph illustrating the average values of the amounts of change in the biological indices for the individual factors for stress plotted in FIG. 1;

FIG. 3 is a diagram illustrating an example of a schematic configuration of a stress evaluation device according to a first embodiment;

FIG. 4 is a diagram illustrating a specific example of the configuration of the stress evaluation device based on the configuration illustrated in FIG. 3;

FIG. 5 is a flowchart for describing a stress evaluation method according to the first embodiment;

FIG. 6 is a diagram illustrating an example of heartbeat information acquired by the stress evaluation device according to the first embodiment;

FIG. 7 is a diagram for describing a method for calculating the amount of variation in heartbeat intervals (RRIs);

FIG. 8 is a diagram for describing an example of use of the stress evaluation device according to the first embodiment;

FIG. 9A is a graph that plots the amounts of change in biological indices of twenty subjects for individual factors for stress;

FIG. 9B is a graph corresponding to FIG. 9A viewed from the positive side of the axis indicating the amount of change in RRI;

FIG. 9C is a graph corresponding to FIG. 9A viewed from the negative side of the axis indicating the amount of change in CvRR;

FIG. 9D is a graph corresponding to FIG. 9A viewed from the negative side of the axis indicating the amount of change in SC;

FIG. 10A is a graph illustrating the average values of the amounts of change in the biological indices for the individual factors for stress plotted in FIG. 9A;

FIG. 10B is a graph corresponding to FIG. 10A viewed from the positive side of the axis indicating the amount of change in RRI;

FIG. 100 is a graph corresponding to FIG. 10A viewed from the negative side of the axis indicating the amount of change in CvRR;

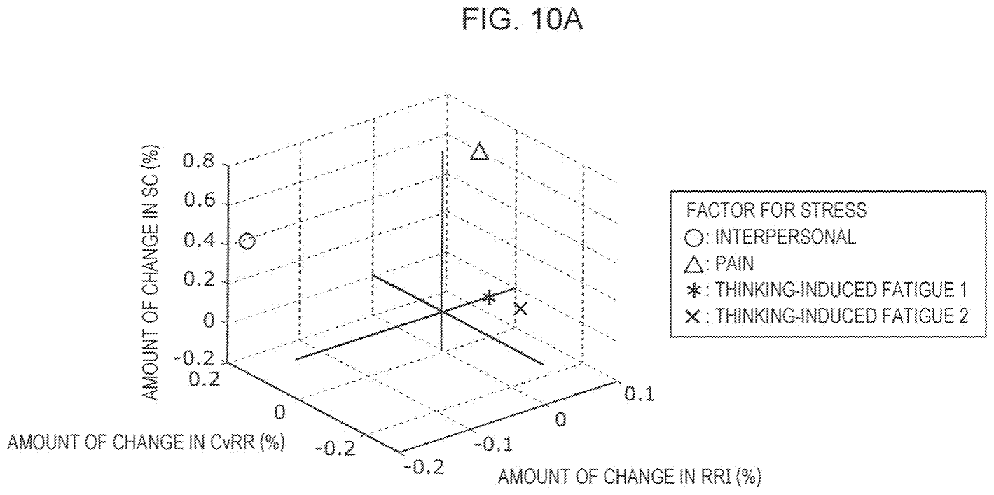

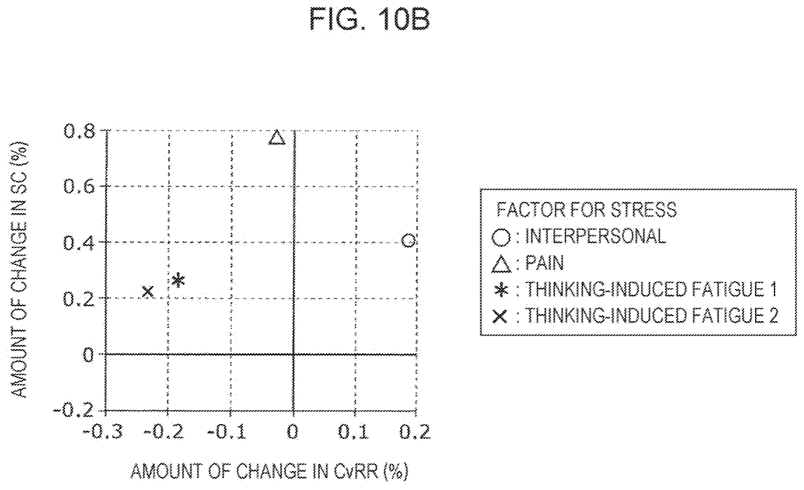

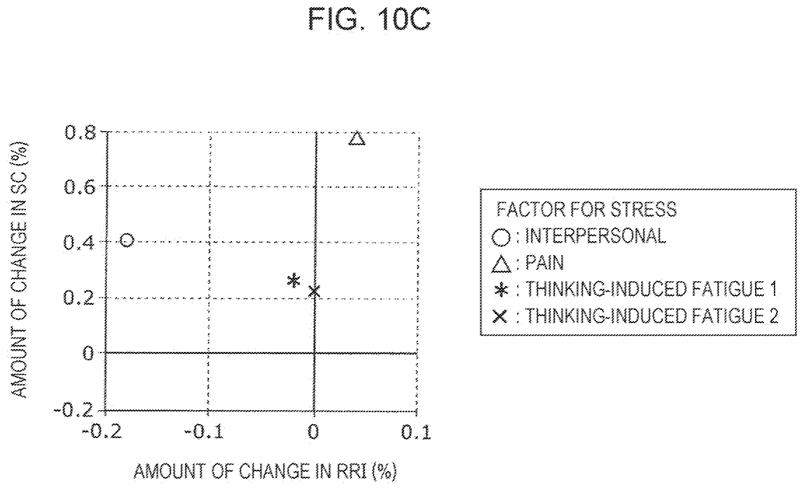

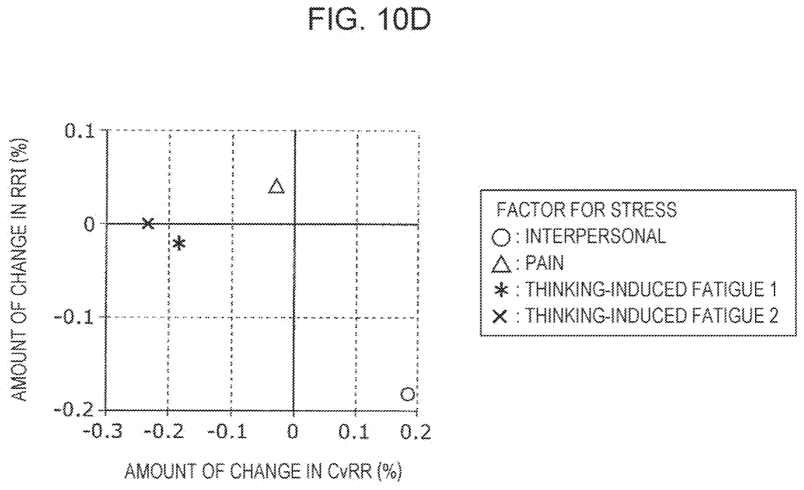

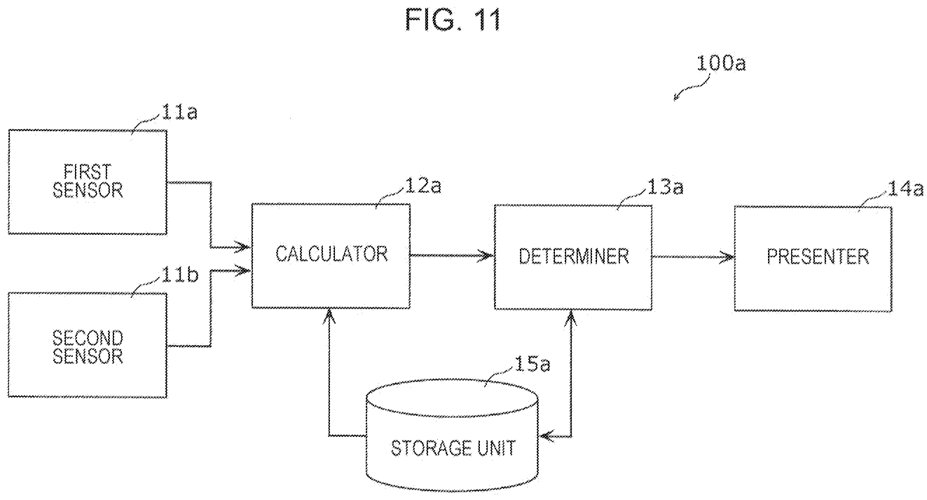

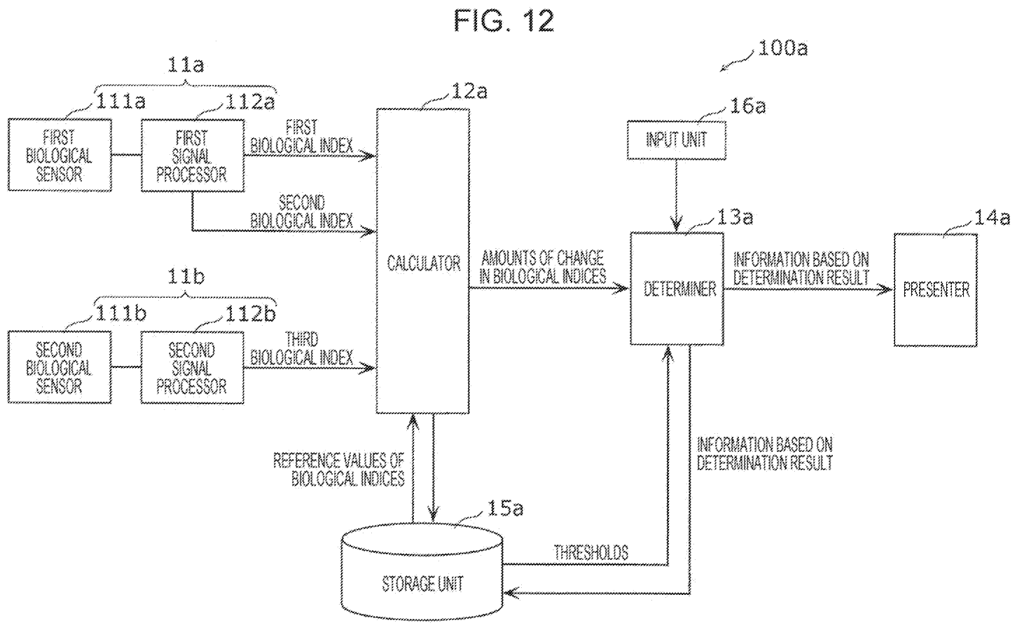

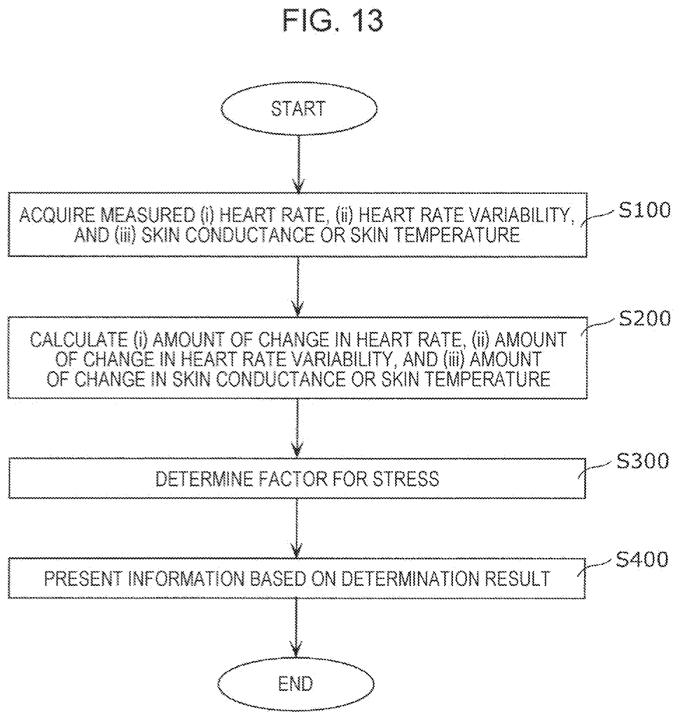

FIG. 10D is a graph corresponding to FIG. 10A viewed from the negative side of the axis indicating the amount of change in SC;

FIG. 11 is a diagram illustrating an example of a schematic configuration of a stress evaluation device according to a second embodiment;

FIG. 12 is a diagram illustrating a specific example of the configuration of the stress evaluation device based on the configuration illustrated in FIG. 11;

FIG. 13 is a flowchart for describing a stress evaluation method according to the second embodiment; and

FIG. 14 is a diagram for describing an example of use of the stress evaluation device according to the second embodiment.

### DETAILED DESCRIPTION

First Underlying Knowledge Forming Basis of the Present Disclosure

Stress disorders in modern society, such as depression, often become severe because of stress accumulated in everyday life. To avoid such a problem, it is important to reduce the accumulation of stress in daily life. In other words, it is desired that people be able to control their stress states. For this purpose, it is desired to sense the state of stress of a user in daily life and provide the user with measures for reducing the stress, such as an appropriate stress relief method and an appropriate stress avoidance method, in accordance with the intensity of the stress and the factor for the stress.

For example, the stress determination system described in Patent Document 1 calculates an activity intensity or the like of a measurement subject on the basis of information acquired from an acceleration sensor and determines a stress state of the measurement subject on the basis of the activity intensity and a biological index, such as heart rate, heartbeat waveform, blood pressure, blood oxygen saturation, body temperature, or perspiration level. This system measures the biological index only when the activity intensity is lower than or equal to a predetermined value, thereby determining a stress state of the measurement subject in daily life.

However, although the stress determination system described in Patent Document 1 is capable of determining the presence or absence of stress, the system is incapable of acquiring information about a factor for stress. There are various factors for stress to which people are subjected. In addition, an optimum stress relief method and an optimum stress avoidance method vary according to a factor for stress. The stress determination system described in Patent Document 1 is incapable of acquiring information about a factor for stress, and thus the system is incapable of providing a user with an appropriate stress relief method and an appropriate stress avoidance method and is insufficient for controlling the stress of the user.

The life support device described in Patent Document 2 acquires action information of a measurement subject as well as biological information, such as an electrocardiogram and pulse waves, analyzes and determines a situation around the measurement subject, and provides the measurement subject with a stress relief method or the like.

However, it is difficult for the life support device described in Patent Document 2 to determine a factor for stress actually felt by a measurement subject because a factor for stress may vary among measurement subjects even when the situations around the measurement subjects are the same. Thus, the life support device described in Patent Document 2 has a risk of presenting an inappropriate stress relief method and an inappropriate stress coping action to the measurement subject.

The inventors earnestly conducted a study in view of the foregoing issues. The details of the study are as follows.

The inventors conducted the following monitoring tests to determine the relationship between factors for stress and a plurality of types of biological indices acquired from biological information, such as heartbeat information.

Monitoring Tests

Four tasks related to different factors for stress were given to each of twenty subjects, and biological signals of the subjects performing the tasks were measured.

As the subjects twenty people who were male or female working adults or university students in their twenties to thirties and who did not show abnormal values in the results of questionnaires about their health and mental states were selected.

The four tasks were [1] a task related to stress from an interpersonal relationship, [2] a task related to stress from pain, [3] a task related to stress from thinking-induced fatigue 1, and [4] a task related to stress from thinking-induced fatigue 2. These tasks were carried out by each subject. The details of each task are as follows.

[1] Task Related to Stress from Interpersonal Relationship

Two task explainers who were strangers to the subject, one being a man and the other being a woman, explained the task to the subject, asked the subject to perform the task, and measured biological signals of the subject performing the task. Specifically, the task explainers told the subject that a mock job interview would be held after 5 minutes and that the subject was to decide what to speak about within 5 minutes before the start of the interview. Measurement of biological signals was performed for the 5 minutes during which the subject was preparing what to speak about, in consideration of gestures and noise of a conversation.

[2] Task Related to Stress from Pain

Electrical stimulations adjusted to give sufficient pain to the subject were given to a forearm of the subject for 10 minutes. The electrical stimulations were randomly given about 10 times per minute, and this procedure was repeatedly performed for 10 minutes. Measurement of biological signals was performed for the first 5 minutes from the start of giving the electrical stimulations.

[3] Task Related to Stress from Thinking-Induced Fatigue 1

The subject was asked to answer two-digit or three-digit multiplication questions displayed on a display within a time limit. The subject performed mental arithmetic on each multiplication question and selected an answer from among three choices displayed on the display. The difficulty levels of the questions and the time limit per question were determined by measuring in advance the mental arithmetic ability of the subject. The subject performed this task for 15 minutes. Measurement of biological signals was performed for the first 5 minutes from when the subject started the task.

(4) Task Related to Stress from Thinking-Induced Fatigue 2

The subject was asked to select a correct answer, to each of paper-rock-scissors questions output from a speaker, from among three choices displayed on a display within a time limit. The time limit per question was determined by measuring in advance the answering ability of the subject. The subject performed this task for 15 minutes. Measurement of biological signals was performed for the first 5 minutes from when the subject started the task.

The above monitoring tests were conducted on the individual subjects at the same time on different days in consideration of diurnal variation.

At-rest biological signals of the subject were biological signals measured for 5 minutes at the same position as that for performing a task before execution of each of the tasks [1] to [4]. Biological indices were calculated from the biological signals and set as reference values for calculating the amounts of change in biological indices. The amounts of change in biological indices are biological indices calculated from the biological signals of the subject measured during a task relative to the at-rest biological indices of the subject.

The measured biological signals were an electrocardiogram (ECG), breathing interval, fingertip skin temperature (SKT), and fingertip skin conductance (SC). These biological signals were measured simultaneously. A plurality of types of biological indices were acquired from each biological signal. Hereinafter, a result of consideration using the ECG will be described.

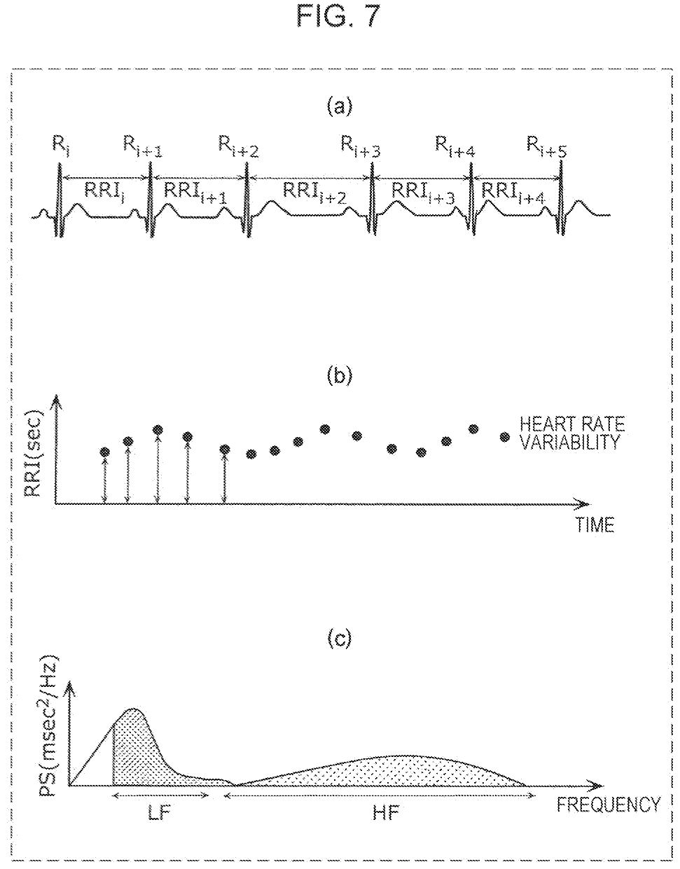

Heartbeat intervals (R-R intervals (RRIs)), each being an interval between the peaks of R waves of two consecutive heartbeats, were calculated from the measured ECG (see FIG. 7(a)). The RRI is one of the indices of heart rate. Furthermore, a coefficient of variation of R-R intervals (CvRR) was calculated from the calculated RRIs. The CvRR is one of the indices of heart rate variability. The CvRR was calculated from the RRIs by normalizing a standard deviation SD of the RRIs in a certain time period by using an average value of the RRIs in the certain time period, as expressed by the following equation (1).

CvRR=SD of heartbeat intervals in certain time period/average of heartbeat intervals in certain time period Equation (1)

In addition, the consecutive RRIs were converted into the relationship between two axes, time and RRI, which was further converted into regular-interval chronological data of RRI (see FIG. 7(b)). Next, frequency analysis was performed by using fast Fourier transform (FFT) (see FIG. 7(c)). Accordingly, a high frequency (HF) and a low frequency (LF) serving as biological indices indicating frequency components of heart rate variability were calculated. The HF and LF are indices of heart rate variability. The HF is an integral of a power spectrum in a high-frequency region from 0.14 Hz to 0.4 Hz and is considered to reflect the amount of parasympathetic nerve activity. The LF is an integral of a power spectrum in a low-frequency region from 0.04 Hz to 0.14 Hz and is considered to reflect the amount of sympathetic nerve activity and parasympathetic nerve activity. The data subjected to frequency analysis using FFT was data of heart rate variability for 60 seconds, and the frequency analysis was performed at intervals of 5 seconds.

The at-rest biological index of the subject and the biological index measured while the subject was performing a task are each an average value of the biological index for 240 seconds after 60 seconds from the start of measurement. The amount of change in a biological index is the amount of change from a reference value that is an average value of the at-rest biological index of the subject to an average value of the biological index measured while the subject is performing the task. The amount of change is expressed as a ratio or a difference. In a case where the amount of change in a biological index is expressed as a ratio, the amount of change in the biological index is calculated by using the following equation (2).

Amount of change in biological index=(average value of biological index during task−average value of at-rest biological index)/average value of at-rest biological index Equation (2)

Subsequently, a combination of the amounts of change in biological indices having high performance to determine a factor for stress was considered. Specifically, linear discriminant analysis was performed by using the calculated amounts of change in RRI, CvRR, LF, and HF.

As a result of performing linear discriminant analysis using the amounts of change in RRI and CvRR, the determination accuracy was 75.0%. Accordingly, it was found that the use of the amount of change in RRI and the amount of change in CvRR makes it possible to determine a factor for stress with relatively high accuracy.

In addition, as a result of performing linear discriminant analysis using the amounts of change in RRI, LF, and HF, the determination accuracy was 67.5%. Accordingly, it was found that the use of the amount of change in RRI, the amount of change in LF, and the amount of change in HF makes it possible to determine a factor for stress with relatively favorable accuracy.

On the other hand, as a result of performing linear discriminant analysis using the amounts of change in LF and HF, the determination accuracy was 46.3%. In other words, when the amount of change in LF and the amount of change in HF were used, the determination accuracy significantly decreased compared to the combination including the amount of change in RRI. From the above consideration, it was found that the use of the amount of change in RRI and the amount of change in CvRR makes it possible to determine a factor for stress with relatively high accuracy.

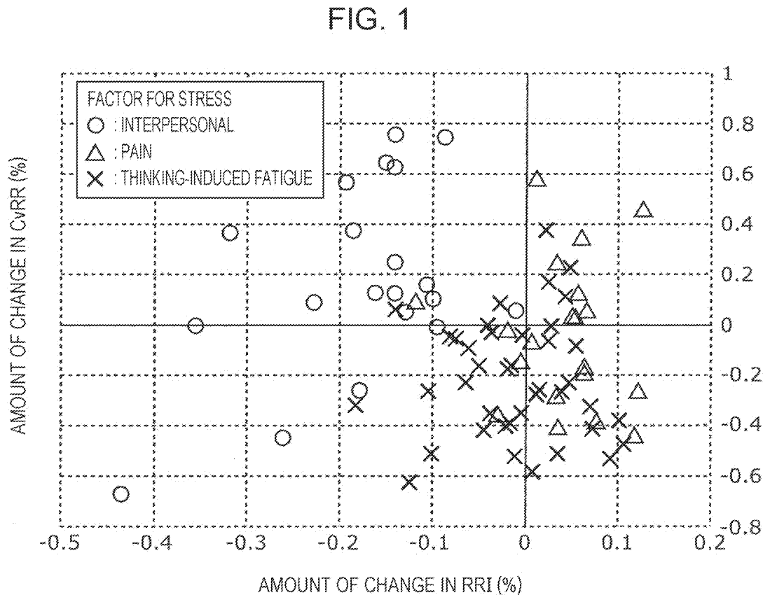

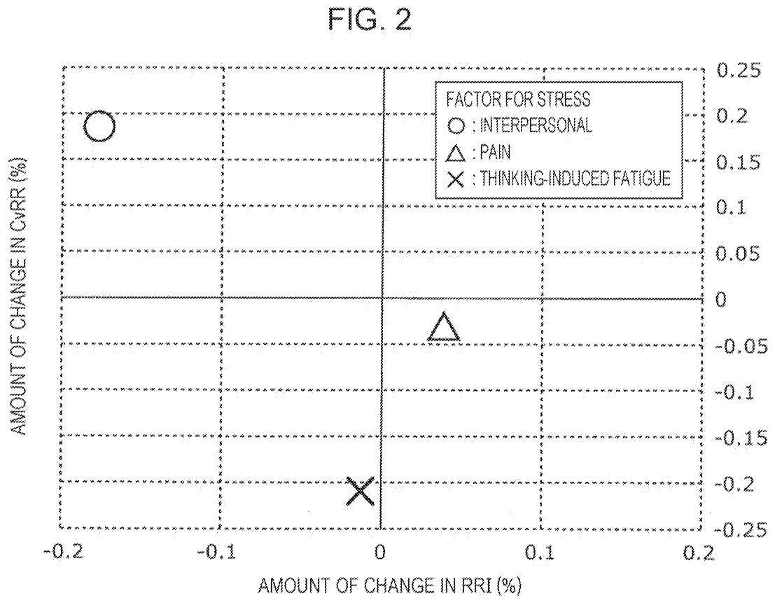

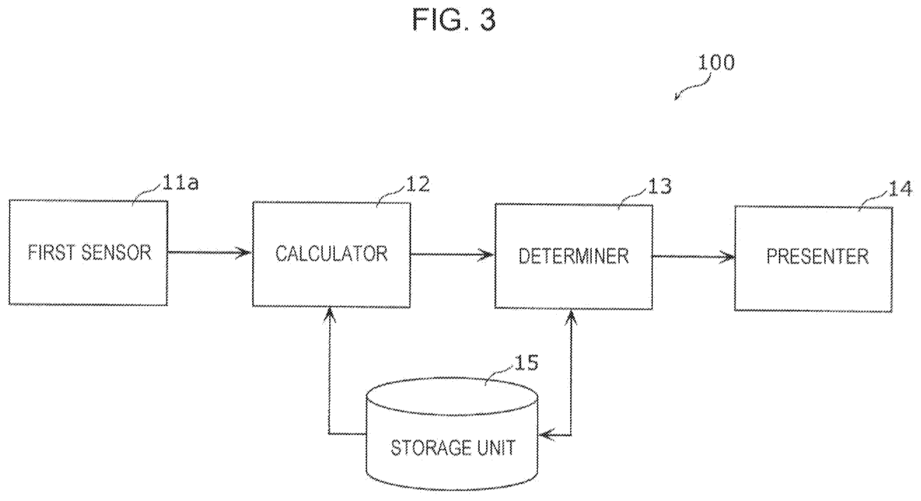

Therefore, factors for stress were determined by using the amount of change in RRI and the amount of change in CvRR as the amounts of change in biological indices. FIG. 1 is a graph that plots the amounts of change in the biological indices of the twenty subjects for individual factors for stress. Stress from thinking-induced fatigue 1 and stress from thinking-induced fatigue 2 are collectively illustrated as stress from thinking-induced fatigue because both results were similar to each other. It was found from FIG. 1 that the trends in the amounts of change in the biological indices vary according to the type of the task that is performed. To make the trends in the changes clearer, average values of the amounts of change in the biological indices of the twenty subjects were calculated. FIG. 2 is a graph illustrating the average values of the amounts of change in the biological indices of the twenty subjects for the individual factors for stress. It was found from FIG. 2 that the amounts of change in the biological indices have the following characteristic trends according to the factors for stress.

In a case where the factor for stress is an interpersonal-related factor, there is a trend that the amount of change in RRI significantly shifts to the negative side (i.e., the heart rate increases) and the amount of change in CvRR shifts to the positive side. In a case where the factor for stress is pain, there is a trend that the amount of change in RRI shifts to the positive side (i.e., the heart rate decreases) and the amount of change in CvRR slightly shifts to the negative side. In a case where the factor for stress is thinking-induced fatigue, there is a trend that the amount of change in RRI very slightly shifts to the negative side (i.e., the heart rate hardly changes) and the amount of change in CvRR significantly shifts to the negative side.

From the above results, it was found that the use of the amount of change in RRI and the amount of change in CvRR makes it possible to determine a factor for stress with relatively high accuracy. It was also found that the trends in the amount of change in RRI and the amount of change in CvRR vary according to a factor for stress. It was further found that a factor for stress of a subject can be easily and accurately determined on the basis of the trends in the amounts of change.

As a result of the above consideration, the inventors have acquired the knowledge that the amount of change in each biological index has a predetermined trend according to a factor for stress and particularly that a factor for stress can be determined more accurately by using both the amounts of change in biological indices related to heart rate and heart rate variability as indices for determination than by using either one of them as an index for determination. In addition, on the basis of the result of the consideration, the inventors have conceived of a device that determines a factor for stress of a measurement subject and an intensity of the stress by comparing the amounts of change in a plurality of types of biological indices acquired from the measurement subject with thresholds.

Accordingly, one embodiment of the present disclosure provides a stress evaluation device, a stress evaluation method, and a non-transitory computer-readable medium that are capable of determining a factor for stress of a measurement subject.

An overview of one aspect of the present disclosure is as follows.

A stress evaluation device according to one aspect of the present disclosure includes a first sensor that measures a heart rate and a heart rate variability of a measurement subject; a calculator that calculates (i) an amount of change in heart rate and (ii) an amount of change in heart rate variability; and a determiner that determines a factor for stress of the measurement subject in accordance with (i) the amount of change in heart rate and (ii) the amount of change in heart rate variability and that outputs information based on a determination result. The amount of change in heart rate is an amount of change from a reference value that is an at-rest heart rate of the measurement subject to the heart rate measured by the first sensor. The amount of change in heart rate variability is an amount of change from a reference value that is an at-rest heart rate variability of the measurement subject to the heart rate variability measured by the first sensor. The determiner makes (I) a comparison between relative magnitudes of the amount of change in heart rate and a first threshold, and (II) a comparison between relative magnitudes of the amount of change in heart rate variability and a second threshold to determine the factor for the stress.

With the above configuration, the amounts of change in individual biological indices are calculated on the basis of at-rest biological indices of the measurement subject, and thus transition of the individual biological indices can be grasped more accurately. Thus, as a result of comparing the relative magnitudes of the amounts of change in individual biological indices and the thresholds of the individual biological indices, the factor for the stress can be determined.

For example, in the stress evaluation device according to the one aspect of the present disclosure, the amount of change in heart rate may be an amount of change to the heart rate measured at a first time, the amount of change in heart rate variability may be an amount of change to the heart rate variability measured at a second time, the first threshold may be the heart rate measured at a certain time different from the first time and the second time relative to the at-rest heart rate of the measurement subject, and the second threshold may be the heart rate variability measured at the certain time relative to the at-rest heart rate variability of the measurement subject.

Here, the certain time is, for example, a time before the measurement subject feels stress. Accordingly, the first threshold and the second threshold can be accurately set. For example, in the case of comparing the relative magnitudes of the amounts of change in individual biological indices and the thresholds, biological indices measured at a predetermined time during sleep or immediately before bedtime of the measurement subject may be set as the thresholds of the individual biological indices. Accordingly, the thresholds can be set in consideration of menstrual variation of women, interannual variability, or the like without setting the certain time by the measurement subject, and thus the factor for the stress can be determined more accurately.

For example, in the stress evaluation device according to the one aspect of the present disclosure, the heart rate variability may be obtained by performing frequency analysis on heartbeat intervals of the measurement subject.

Accordingly, the stress evaluation device is capable of acquiring information about a breathing interval and a blood pressure from the frequency components of heart rate variability. Thus, the stress evaluation device is capable of using biological indices including detailed information of the measurement subject as indices (determination indices) for determining stress, and is thus capable of determining the factor for the stress of the measurement subject more accurately.

For example, in the stress evaluation device according to the one aspect of the present disclosure, in a case where the amount of change in heart rate is larger than the first threshold and the amount of change in heart rate variability is larger than the second threshold, the determiner may determine that the factor for the stress is an interpersonal-related factor.

With the above configuration, as a result of comparing the relative magnitudes of the amounts of change in individual biological indices and the thresholds of the individual biological indices, it can be determined that the factor for the stress is an interpersonal-related factor.

For example, in the stress evaluation device according to the one aspect of the present disclosure, in a case where the amount of change in heart rate is larger than the first threshold and the amount of change in heart rate variability is smaller than the second threshold, the determiner may determine that the factor for the stress is pain.

With the above configuration, as a result of comparing the relative magnitudes of the amounts of change in individual biological indices and the thresholds of the individual biological indices, it can be determined that the factor for the stress is pain.

For example, in the stress evaluation device according to the one aspect of the present disclosure, in a case where the amount of change in heart rate is smaller than the first threshold and the amount of change in heart rate variability is larger than the second threshold, the determiner may determine that the factor for the stress is thinking-induced fatigue.

With the above configuration, as a result of comparing the relative magnitudes of the amounts of change in individual biological indices and the thresholds of the individual biological indices, it can be determined that the factor for the stress is thinking-induced fatigue.

For example, in the stress evaluation device according to the one aspect of the present disclosure, the determiner may further determine an intensity of the stress in accordance with a difference between the amount of change in heart rate and the first threshold and a difference between the amount of change in heart rate variability and the second threshold, and may output a determination result as the information based on the determination result.

Accordingly, the measurement subject is able to know the intensity of his/her stress. Accordingly, the measurement subject is able to be aware of stress control more easily and grasp the trend in his/her stress more easily. For example, the measurement subject is able to recognize that the tolerable stress intensity varies among a plurality of types of factors for stress. Accordingly, the measurement subject becomes able to determine whether stress control is immediately necessary in accordance with the condition of stress. Thus, the measurement subject is able to efficiently control stress and is thus able to continuously control stress.

For example, the stress evaluation device according to the one aspect of the present disclosure may further include a presenter that presents the information based on the determination result output by the determiner. The information may include at least one selected from the group consisting of the factor for the stress, an intensity of the stress, and measures for reducing the stress.

Accordingly, the measurement subject is able to know his/her stress condition and a stress control method immediately after feeling stress, and is thus able to reduce accumulation of stress.

For example, in the stress evaluation device according to the one aspect of the present disclosure, the presenter may present the information by using a sound.

Accordingly, the measurement subject is able to easily know his/her stress condition and a stress control method in daily life, and is thus able to keep awareness about control of his/her stress more easily. Thus, the measurement subject is able to continuously control his/her stress.

For example, in the stress evaluation device according to the one aspect of the present disclosure, the presenter may present the information by using an image.

Accordingly, the measurement subject is able to visually grasp his/her stress condition and a stress control method, and is thus able to clearly be aware of control of his/her stress. Thus, the measurement subject is able to continuously control his/her stress.

A stress evaluation method according to one aspect of the present disclosure includes acquiring a measured heart rate and a measured heart rate variability of a measurement subject; calculating (i) an amount of change in heart rate and (ii) an amount of change in heart rate variability; and determining a factor for stress of the measurement subject in accordance with (i) the amount of change in heart rate and (ii) the amount of change in heart rate variability and outputting information based on a determination result. The amount of change in heart rate is an amount of change from a reference value that is an at-rest heart rate of the measurement subject to the measured heart rate. The amount of change in heart rate variability is an amount of change from a reference value that is an at-rest heart rate variability of the measurement subject to the measured heart rate variability. The determining includes making (I) a comparison between relative magnitudes of the amount of change in heart rate and a first threshold, and (II) a comparison between relative magnitudes of the amount of change in heart rate variability and a second threshold to determine the factor for the stress.

With the above method, the amounts of change in individual biological indices are calculated on the basis of at-rest biological indices of the measurement subject, and thus transition of the individual biological indices can be grasped more accurately. Thus, as a result of comparing the relative magnitudes of the amounts of change in individual biological indices and the thresholds of the individual biological indices, the factor for the stress can be determined.

It should be noted that general or specific embodiments may be implemented as a system, a device, a method, an integrated circuit, a computer program, a computer-readable recording medium such as a CD-ROM, or any selective combination thereof.

Hereinafter, embodiments of the present disclosure will be described in detail with reference to the attached drawings.

The embodiments described below all illustrate general or specific examples. The numerical values, shapes, components, layout positions and connection states of components, steps, and the order of steps described in the following embodiments are merely examples, and are not intended to limit the present disclosure. In addition, among the components in the following embodiments, a component that is not described in an independent claim indicating the broadest concept will be described as an optional component. Each figure is not necessarily strictly illustrated. In the individual figures, substantially the same components are denoted by the same reference numerals, and a duplicate description may be omitted or simplified.

### First Embodiment

Hereinafter, a stress evaluation device, a stress evaluation method, and a non-transitory computer-readable medium according to a first embodiment will be described by using specific examples.

Overview of Stress Evaluation Device

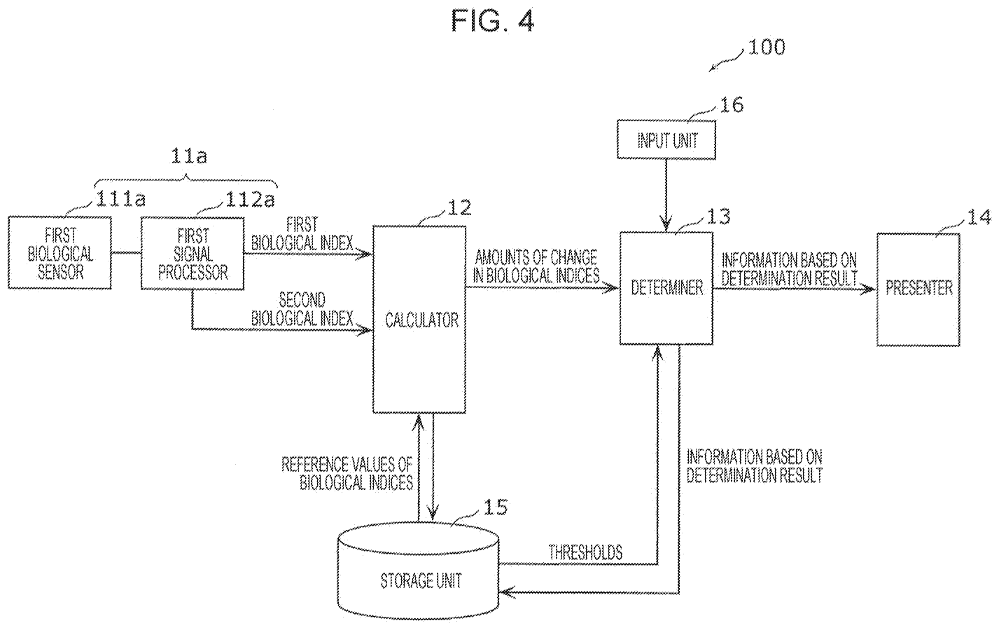

FIG. 3 is a diagram illustrating a schematic configuration of a stress evaluation device 100 according to the first embodiment. As illustrated in FIG. 3, the stress evaluation device 100 includes a first sensor 11a, a calculator 12, a determiner 13, a presenter 14, and a storage unit 15. In the stress evaluation device 100, for example, the first sensor 11a includes a wearable first biological sensor 111a (see FIG. 4) that measures a biological signal of a measurement subject. The first sensor 11a calculates a plurality of types of biological indices from the biological signal measured by the first biological sensor 111a and outputs the calculated biological indices as measured biological indices to the calculator 12. The calculator 12 calculates average values of the individual biological indices of the measurement subject at rest (hereinafter also referred to as reference values) and thresholds of the individual biological indices, and stores the calculated average values and thresholds in the storage unit 15. The calculator 12 also calculates average values of the measured biological indices and the amounts of change in the individual biological indices and outputs the calculated average values and amounts to the determiner 13. The determiner 13 determines a factor for stress of the measurement subject in accordance with the amounts of change in the individual biological indices. More specifically, the determiner 13 compares the relative magnitudes of the amounts of change in the individual biological indices and the thresholds of the individual biological indices to determine a factor for stress. In addition, the determiner 13 determines an intensity of the stress in accordance with the differences between the amounts of change in the individual biological indices and the thresholds of the individual biological indices. Subsequently, the determiner 13 outputs information based on these determination results to the presenter 14. At this time, the determiner 13 stores the information based on the determination results in the storage unit 15. The presenter 14 presents the information based on the determination results. Furthermore, the stress evaluation device 100 may include an input unit 16 (see FIG. 4) for inputting an instruction of the measurement subject (user). In response to an instruction of the measurement subject input to the input unit 16, the determiner 13 causes the presenter 14 to present the information about the determination results.

Configuration of Stress Evaluation Device

The configuration of the stress evaluation device 100 according to the first embodiment will be described in more detail. FIG. 4 is a diagram illustrating a specific example of the configuration of the stress evaluation device 100 based on the configuration illustrated in FIG. 3.

As illustrated in FIG. 4, the stress evaluation device 100 includes the first sensor 11a including the first biological sensor 111a and a first signal processor 112a, the calculator 12, the determiner 13, the presenter 14, the storage unit 15, and the input unit 16.

The first biological sensor 111a measures a biological signal of a measurement subject. The biological signal is a signal of biological information. The biological information is, for example, physiological information affected by stress, such as heartbeats, pulses, the number of breaths, blood oxygen saturation, blood pressure, or body temperature. For easy measurement, the biological information is heartbeat information, for example. The heartbeat information is information acquired from heartbeats. Alternatively, the biological information may be pulse wave information.

The first biological sensor 111a is a sensor that acquires heartbeat information or pulse wave information. In a case where the first biological sensor 111a is a sensor that acquires heartbeat information (hereinafter a heartbeat sensor), the heartbeat sensor is, for example, a sensor including a pair of detection electrodes that are to be in contact with a body surface of a measurement subject. The heartbeat information acquired by the heartbeat sensor is an electric signal acquired from heartbeats and is, for example, an ECG. The heartbeat sensor may include conductive adhesive gel electrodes or dry electrodes formed of conductive fibers. The heartbeat sensor is to be worn on the chest and has, for example, a wearable shape in which wear and electrodes are integrated together.

In a case where the first biological sensor 111a is a sensor that acquires pulse wave information (hereinafter a pulse wave sensor), the pulse wave sensor is, for example, a sensor that measures, with a phototransistor and a photodiode, a change in the amount of blood in blood vessels by using reflected light or transmitted light. The pulse wave sensor measures pulse wave information while being worn around a wrist of a user. The pulse wave sensor may be worn around an ankle, a finger, an upper arm, or the like. The shape of the pulse wave sensor is not limited to a band shape (for example, a wristwatch shape) and may be an attachable shape to be attached to the neck or the like, or an eyeglass shape. Alternatively, the pulse wave sensor may be an image sensor that measures pulse wave information by using a change in chromaticity of skin of the face or a hand and calculates pulses.

The biological signal measured by the first biological sensor 111a is output to the first signal processor 112a.

The first signal processor 112a calculates a plurality of types of biological indices from the one biological signal measured by the first biological sensor 111a. In the first embodiment, two types of biological indices, a first biological index and a second biological index, are calculated. As described above, in a case where the biological signal is an ECG, the plurality of types of biological indices are RRI, CvRR, HF, LF, and the like. The RRI is an index of heart rate, whereas the CvRR, HF, and LF are indices of heart rate variability. Furthermore, the first signal processor 112a may calculate biological indices of variations in the number of breaths and blood pressure from the frequency components of heart rate variability. Among the plurality of types of biological indices, a combination of RRI and CvRR is a combination that achieves relatively high determination accuracy. Thus, in the first embodiment, a description will be given of an example in which the first biological index and the second biological index are RRI and CvRR, respectively. The methods for calculating RRI and CvRR are as described above regarding the monitoring tests. The first signal processor 112a outputs the calculated first biological index and second biological index to the calculator 12.

The calculator 12 acquires the first biological index and second biological index output by the first signal processor 112a and calculates, from the acquired first biological index and second biological index, the amount of change in the first biological index and the amount of change in the second biological index. The amount of change in a biological index is a measured biological index relative to an at-rest biological index of a measurement subject (hereinafter also referred to as a reference value), and is expressed as a difference or a ratio. The reference values of the individual biological indices are stored in the storage unit 15. The calculator 12 reads out the reference values of the first and second biological indices stored in the storage unit 15 and calculates the amounts of change in the first and second biological indices relative to the reference values. The calculator 12 outputs the calculated amounts of change in the individual biological indices to the determiner 13. The reference values may vary according to a season or a menstrual cycle of the measurement subject and thus may be updated at predetermined intervals.

The calculator 12 also calculates the thresholds of the individual biological indices. For example, in a case where the first biological index is heart rate, the amount of change in heart rate is the amount of change to a heart rate measured at a first time. A first threshold is a threshold of the first biological index and is, for example, a threshold of RRI, which is an index of heart rate. The first threshold is a heart rate measured at a certain time relative to an at-rest heart rate of the measurement subject. For example, in a case where the second biological index is heart rate variability, the amount of change in heart rate variability is the amount of change to a heart rate variability measured at a second time. A second threshold is a threshold of the second biological index and is, for example, a threshold of CvRR, which is an index of heart rate variability. The second threshold is a heart rate variability measured at the certain time relative to an at-rest heart rate variability of the measurement subject. In other words, each of these thresholds is the amount of change in the biological index, which is a difference or a ratio between the reference value and the measurement value of the biological index measured at the certain time that is different from the first time and the second time. Here, the certain time is, for example, a time before the measurement subject feels stress.

In the first embodiment, a description will be given of a case where the first time and the second time are the same. Alternatively, the first time and the second time may be different from each other. For example, the first signal processor 112a may calculate a plurality of types of heart rate and heart rate variability in a time-division manner from the one biological signal measured by the first biological sensor 111a. At this time, the calculator 12 calculates the amount of change to the heart rate measured at the first time and calculates the amount of change to the heart rate variability measured at the second time different from the first time.

The calculator 12 reads out the thresholds of the individual biological indices stored in the storage unit 15 and compares the relative magnitudes of the amounts of change in the individual biological indices and the thresholds. Subsequently, the calculator 12 determines a period during which at least one of the amounts of change in the individual biological indices exceeds the threshold for a predetermined time to be a stress occurrence period. The stress occurrence period is a period during which the measurement subject feels stress. The calculator 12 calculates representative values of the amounts of change in the individual biological indices from the amounts of change in the individual biological indices during the stress occurrence period. As the representative values of the amounts of change in the individual biological indices during the stress occurrence period, for example, average values of the amounts of change in the individual biological indices during the stress occurrence period may be used, or values having the largest differences from the reference values (maximum values) may be used.

The determiner 13 acquires the representative values of the amounts of change in the first and second biological indices output by the calculator 12 and reads out the first and second thresholds stored in the storage unit 15. The determiner 13 compares the relative magnitudes of the representative value of the amount of change in the first biological index during the stress occurrence period and the first threshold and also compares the relative magnitudes of the representative value of the amount of change in the second biological index during the stress occurrence period and the second threshold, thereby determining a factor for stress of the measurement subject. In other words, the determiner 13 determines a factor for stress in each stress occurrence period. The representative value of the amount of change in a biological index is an example of the amount of change in the biological index, and thus hereinafter the representative value of the amount of change in a biological index will also be referred to as the amount of change in the biological index.

Specifically, in a case where the amount of change in the first biological index (here, heart rate) is larger than the first threshold and the amount of change in the second biological index (here, heart rate variability) is larger than the second threshold, the determiner 13 determines that a factor for stress is an interpersonal-related factor. In a case where the amount of change in the first biological index is larger than the first threshold and the amount of change in the second biological index is smaller than the second threshold, the determiner 13 determines that a factor for stress is pain. In a case where the amount of change in the first biological index is smaller than the first threshold and the amount of change in the second biological index is larger than the second threshold, the determiner 13 determines that a factor for stress is thinking-induced fatigue.

Furthermore, the determiner 13 determines an intensity of the stress in accordance with the difference between the amount of change in the first biological index and the first threshold and the difference between the amount of change in the second biological index and the second threshold, and outputs a determination result as information based on the determination result. The information based on the determination result includes, for example, at least one of the factor for the stress, the intensity of the stress, or measures for reducing the stress. The measures for reducing the stress may be, for example, a stress relief method or a stress avoidance method. The measures for reducing the stress are included in a presentation information table, which will be described below. The determiner 13 reads out appropriate measures for reducing the stress from the presentation information table stored in the storage unit 15 and outputs the measures to the presenter 14.

In addition, the determiner 13 stores the information based on the determination result in the storage unit 15. At this time, the determiner 13 may store the information based on the determination result in the storage unit 15 in association with information about the time when the measurement subject felt the stress.

The presenter 14 presents the information based on the determination result output by the determiner 13. The presenter 14 may present the information based on the determination result by using a sound or an image. In a case where the presenter 14 presents the information by using a sound, the presenter 14 is, for example, a speaker. In a case where the presenter 14 presents the information by using an image, the presenter 14 is, for example, a display.

The storage unit 15 stores the reference values of the individual biological indices, the thresholds of the individual biological indices, the presentation information table, and the like. The presentation information table is a table of presentation information, such as measures for reducing stress, presented in accordance with a factor for stress and an intensity of the stress. As described above, the reference values and thresholds of the individual biological indices may be updated at predetermined intervals. Also, the presentation information table may be updated at predetermined intervals.

The storage unit 15 also stores the information based on the determination result output by the determiner 13, such as the factor for the stress, the intensity of the stress, and the measures for reducing the stress. At this time, the storage unit 15 may store the information based on the determination result in association with the stress occurrence period. Accordingly, the measurement subject is able to retrieve the information based on the determination result at desired timing. At this time, the determiner 13 causes the presenter 14 to present the information based on the determination result in response to an operation of the measurement subject input by the input unit 16.

The input unit 16 outputs an operation signal indicating the operation performed by the measurement subject to the determiner 13. The input unit 16 is, for example, a keyboard, a mouse, a touch screen, a microphone, or the like. The operation signal is a signal for setting a method for extracting the information based on the determination result or a presentation method in the presenter 14. On the basis of the setting input to the input unit 16, the presenter 14 presents various forms of determination results, for example, a change in stress during a predetermined period, a factor for stress to which the measurement subject is susceptible, and measures for reducing stress suitable for the measurement subject. Accordingly, the measurement subject is able to grasp middle-term and long-term trends in stress as well as a short-term trend in stress. In this way, the measurement subject is able to know effective measures for reducing stress suitable for the measurement subject and is thus able to control the stress in a middle-term or long-term vision.

Stress Evaluation Method

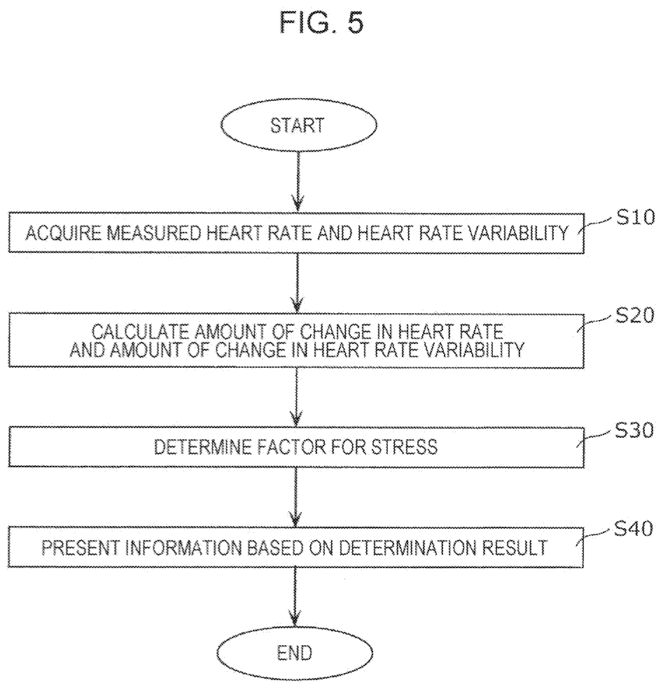

The stress evaluation method according to the first embodiment will be described in detail with reference to FIG. 5. FIG. 5 is a flowchart for describing the stress evaluation method according to the first embodiment.

The stress evaluation method according to the first embodiment includes an acquisition step S10 of acquiring a measured heart rate and a measured heart rate variability of a measurement subject; a calculation step S20 of calculating (i) the amount of change in heart rate and (ii) the amount of change in heart rate variability; and a determination step S30 of determining a factor for stress of the measurement subject in accordance with (i) the amount of change in heart rate and (ii) the amount of change in heart rate variability and outputting information based on a determination result. The amount of change in heart rate is the amount of change from a reference value that is an at-rest heart rate of the measurement subject to the heart rate measured by the first sensor 11a. The amount of change in heart rate variability is the amount of change from a reference value that is an at-rest heart rate variability of the measurement subject to the heart rate variability measured by the first sensor 11a. In the determination step S30, (I) a comparison between relative magnitudes of the amount of change in heart rate and the first threshold, and (II) a comparison between relative magnitudes of the amount of change in heart rate variability and the second threshold are made to determine the factor for the stress. In the first embodiment, the stress evaluation method further includes a presentation step S40 of presenting the information based on the determination result acquired in the determination step S30.

Hereinafter, the individual steps will be described in more detail.

In the acquisition step S10, the calculator 12 acquires a plurality of types of biological indices (here, heart rate and heart rate variability) of the measurement subject measured by the first sensor 11a. In the first sensor 11a, the first biological sensor 111a measures heartbeat information (here, an ECG), and the first signal processor 112a calculates biological indices, such as an index of heart rate and an index of heart rate variability. As described above, the biological information is not limited to heartbeat information and may be physiological information affected by stress, such as pulse wave information. In particular, heartbeat information can be measured in real time more easily and with a smaller burden on the measurement subject than other biological information, such as pulses, the number of breaths, blood pressure, and blood oxygen saturation, when a wearable biological sensor is used. Thus, as a result of using heartbeat information of the measurement subject as biological information, the stress state of the measurement subject can be appropriately evaluated.

The biological indices acquired from the heartbeat information include RRI which is an index of heart rate, and CvRR, LF, HF, and LF/HF which are indices of heart rate variability. In this way, a plurality of types of biological indices can be acquired from one piece of biological information. As described above, a combination of these biological indices makes it possible to determine a factor for stress with relatively high determination accuracy, and thus reliable evaluation can be acquired.

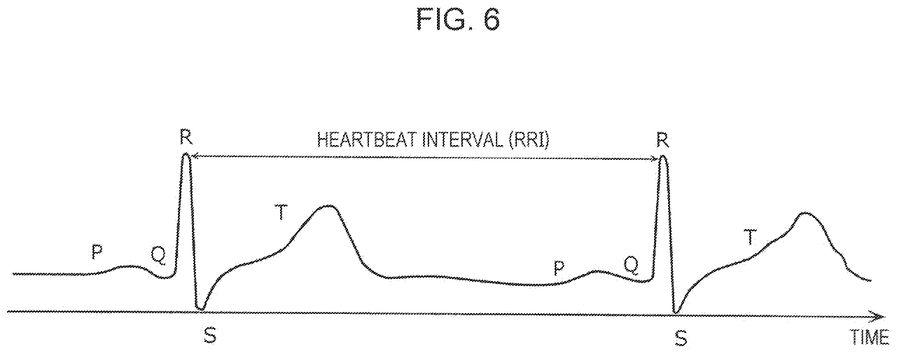

FIG. 6 is a diagram illustrating an example of the heartbeat information acquired by the first sensor 11a of the stress evaluation device 100 according to the first embodiment. The heartbeat information is, for example, an ECG, and is the ECG waveform illustrated in FIG. 6. The ECG waveform is formed of a P wave that reflects electrical excitation of the cardiac atria; a Q wave, an R wave, and an S wave that reflect electrical excitation of the cardiac ventricles; and a T wave that reflects repolarization of excited cardiac myocytes of the ventricles. Among these waves, the R wave has the largest wave height (potential difference) and is the most robust against noise such as a myoelectric potential. Thus, the interval between the peaks of the R waves of two consecutive heartbeats in the ECG waveform, that is, the heartbeat interval (RRI), is calculated. A heart rate is calculated by multiplying the reciprocal of RRI by 60.

Furthermore, as described above regarding the monitoring tests, CvRR is calculated from RRIs by normalizing a standard deviation SD of the RRIs in a certain time period by using an average value of the RRIs in the certain time period, by using the above equation (1).

The first signal processor 112a detects, from the heartbeat information acquired by the first biological sensor 111a, an electric signal (R waves) generated when the left ventricle suddenly contracts to send blood from the heart, and calculates an RRI. To detect R waves, for example, an existing method such as the Pan & Tompkins method is used.

Next, a description will be given of a method for calculating the amount of variation in the heartbeat intervals (RRIs) from the detected R waves by the calculator 12.

FIG. 7 is a diagram for describing a method for calculating the amount of variation in the heartbeat intervals (RRIs). The first signal processor 112a calculates the amount of variation in the RRIs from acquired detection data of R waves in the following manner.

As illustrated in FIG. 7(a), the first signal processor 112a calculates RRIs, each being an interval between the peaks of R waves of two consecutive heartbeats. The first signal processor 112a converts the calculated RRIs into the relationship between two axes, time and RRI. The converted data is discrete data of irregular intervals, and thus the calculator 12 converts the RRI chronological data into the regular-interval chronological data illustrated in FIG. 7(b). Next, the calculator 12 performs frequency analysis on the regular-interval chronological data by using fast Fourier transform (FFT), thereby acquiring frequency components of heart rate variability illustrated in FIG. 7(c).

The frequency components of heart rate variability can be divided into, for example, a high-frequency component HF and a low-frequency component LF. As described above regarding the monitoring tests, the HF is considered to reflect the amount of parasympathetic nerve activity, and the LF is considered to reflect the amount of sympathetic nerve activity and parasympathetic nerve activity. Thus, LF/HF, which is the ratio of LF to HF, is considered to indicate the amount of sympathetic nerve activity.

In this way, the first sensor 11a calculates a plurality of types of biological indices from heartbeat information.

In the acquisition step S10, the calculator 12 acquires two types of biological indices (here, heart rate and heart rate variability) from among these biological indices.

Subsequently, in the calculation step S20, the calculator 12 calculates the amounts of change in the two types of biological indices acquired in the acquisition step S10. As described above, the amount of change in each biological index is acquired by calculating the ratio or difference between the acquired value of the biological index and the reference value of the biological index, which is the value of the at-rest biological index of the measurement subject. The calculator 12 reads out and uses the reference values of the individual biological indices stored in the storage unit 15.

In a case where the amount of change in each biological index is expressed as a difference, for example, the amount of change is calculated by subtracting the reference value of the biological index from the value of the biological index acquired in the acquisition step S10. For example, the amount of change in heart rate is calculated by subtracting the reference value of heart rate from the value of heart rate of the measurement subject acquired in the acquisition step S10. In a case where the amount of change in each biological index is expressed as a ratio, the amount of change is calculated by dividing the value of the biological index acquired in the acquisition step S10 by the reference value of the biological index. For example, the amount of change in heart rate is calculated by dividing the value of heart rate of the measurement subject acquired in the acquisition step S10 by the reference value of heart rate.

In the above-described manner, the calculator 12 calculates the amounts of change in the individual biological indices in the calculation step S20.

Subsequently, in the determination step S30, the determiner 13 determines a factor for stress in accordance with the amounts of change in the individual biological indices calculated in the calculation step S20. The determiner 13 compares the relative magnitudes of the amounts of change in the individual biological indices and the thresholds of the individual biological indices to determine a factor for stress of the measurement subject. Specifically, in the determination step S30, in a case where the amount of change in heart rate is larger than the first threshold and the amount of change in heart rate variability is larger than the second threshold, the determiner 13 determines that a factor for stress is an interpersonal-related factor. In a case where the amount of change in heart rate is larger than the first threshold and the amount of change in heart rate variability is smaller than the second threshold, the determiner 13 determines that a factor for stress is pain. In a case where the amount of change in heart rate is smaller than the first threshold and the amount of change in heart rate variability is larger than the second threshold, the determiner 13 determines that a factor for stress is thinking-induced fatigue.

Furthermore, the determiner 13 determines an intensity of the stress in accordance with the difference between the amount of change in heart rate and the first threshold and the difference between the amount of change in heart rate variability and the second threshold, and outputs a determination result as information based on the determination result.

The first threshold is the threshold of heart rate and is a heart rate measured at the certain time different from the first time and the second time relative to the at-rest heart rate of the measurement subject. The second threshold is the threshold of heart rate variability and is a heart rate variability measured at the certain time different from the first time and the second time relative to the at-rest heart rate variability of the measurement subject. These thresholds are calculated by the calculator 12 and stored in the storage unit 15. The determiner 13 reads out and uses the thresholds of the individual biological indices stored in the storage unit 15. As described above, the certain time is a time before the measurement subject feels stress.

As the threshold of each biological index, a threshold in a case where the amount of change in the biological index is a positive value and a threshold in a case where the amount of change in the biological index is a negative value are set. The reference value corresponds to zero amount of change. The relative magnitudes of the amount of change in each biological index and the threshold are compared in the following manner. In a case where the amount of change in the biological index is a positive value, the relative magnitudes of the amount of change in the biological index and the positive threshold are compared with each other. In a case where the amount of change in the biological index is a negative value, the relative magnitudes of the absolute value of the amount of change in the biological index and the absolute value of the negative threshold are compared with each other. The threshold of each biological index may be a fixed value, may be updated at predetermined intervals, or may be updated on the basis of daily measurements.

The threshold may be calculated by relatively simple machine learning, such as linear discrimination or a decision tree. Accordingly, a determination reference value and a threshold suitable for the measurement subject can be set, and thus a factor for stress can be determined more accurately.

As described above, in the determination step S30, the relative magnitudes of the amounts of change in the individual biological indices and the thresholds of the individual biological indices are compared with each other, and accordingly a factor for stress of the measurement subject is determined.

Subsequently, in the presentation step S40, the presenter 14 presents information based on the determination result obtained by the determiner 13. The presenter 14 may present the information based on the determination result by using a sound or an image. The information based on the determination result includes at least one of a factor for stress, an intensity of stress, or measures for reducing stress. The presenter 14 displays various forms of determination results on the basis of the setting input by the measurement subject in the input unit 16.

Example of Use of Stress Evaluation Device

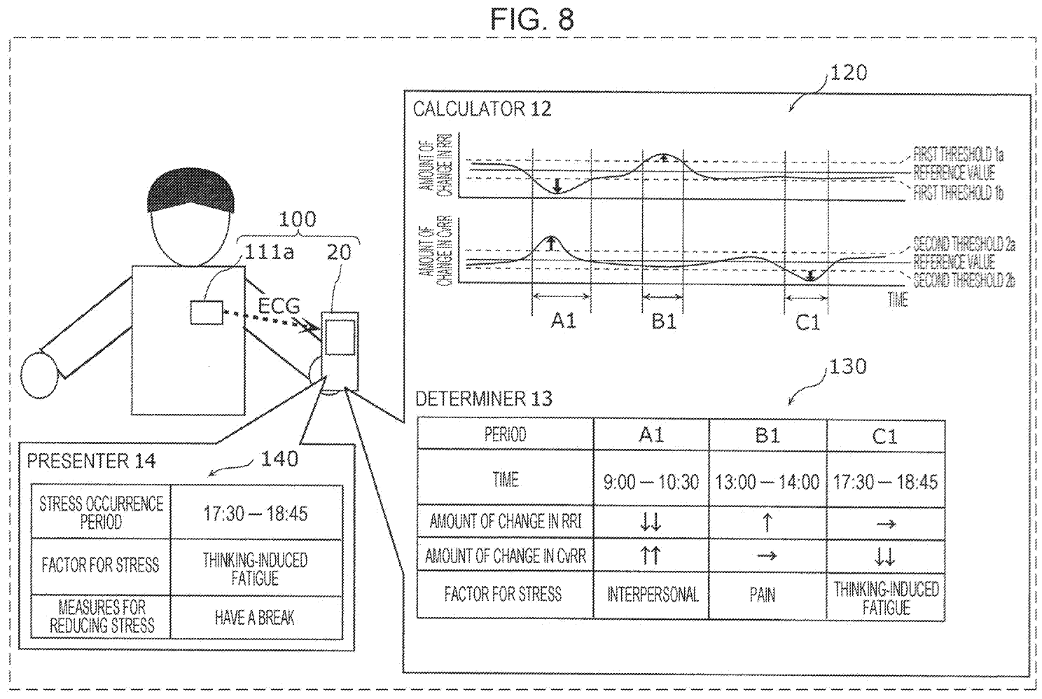

An example of use of the stress evaluation device 100 according to the first embodiment will be described in detail. FIG. 8 is a diagram for describing an example of use of the stress evaluation device 100 according to the first embodiment.

As illustrated in FIG. 8, the stress evaluation device 100 includes the first biological sensor 111a, which is a part of the first sensor 11a, and an evaluation terminal 20 including the components other than the first biological sensor 111a. A measurement subject wears the first biological sensor 111a such that the first biological sensor 111a is in contact with the skin of the chest and measures an ECG. The first biological sensor 111a may include conductive adhesive gel electrodes or dry electrodes formed of conductive fibers. The first biological sensor 111a transmits a measured electric signal of heartbeats to the evaluation terminal 20 through communication. The communication method may be wireless communication using Bluetooth (registered trademark) or the like, or may be wired communication.

The evaluation terminal 20 includes the first signal processor 112a of the first sensor 11a, the calculator 12, the determiner 13, the presenter 14, the storage unit 15, and the input unit 16. The first signal processor 112a receives the electric signal of heartbeats transmitted by the first biological sensor 111a through communication. The first signal processor 112a calculates, from the received electric signal of heartbeats, RRI which is an index of heart rate and CvRR which is an index of heart rate variability, and outputs these biological indices to the calculator 12.

The calculator 12 acquires the RRI and CvRR output by the first signal processor 112a and reads out the reference value of RRI and the reference value of CvRR stored in the storage unit 15. The calculator 12 calculates the amounts of change in these biological indices relative to the read out reference values. The amount of change in each biological index is expressed as a difference or a ratio. In the first embodiment, the amount of change is expressed as a ratio.

As described above, the calculator 12 calculates the thresholds of the individual biological indices and outputs the thresholds to the storage unit 15. As the threshold of each biological index, a threshold in a case where the amount of change in the biological index is a positive value and a threshold in a case where the amount of change in the biological index is a negative value are set. The reference value corresponds to zero amount of change. Specifically, in a case where the amount of change in each biological index is a positive value, the positive threshold is a value larger than the reference value, and corresponds to a first threshold 1a (hereinafter a positive threshold 1a) and a second threshold 2a (hereinafter a positive threshold 2a) in an amount-of-change graph 120. In a case where the amount of change in each biological index is a negative value, the negative threshold is a value smaller than the reference value, and corresponds to a first threshold 1b (hereinafter a negative threshold 1b) and a second threshold 2b (hereinafter a negative threshold 2b) in the amount-of-change graph 120. In addition, the calculator 12 calculates the reference values of the individual biological indices and outputs the reference values to the storage unit 15. The reference value of each biological index corresponds to zero amount of change in the biological index. For example, in the amount-of-change graph 120, the solid line between the positive threshold 1a and the negative threshold 1b corresponds to the reference value. The positive threshold and the negative threshold may or may not be set at regular intervals with the reference value (zero amount of change) interposed therebetween. These thresholds may be appropriately set in accordance with the amount of change in each biological index.

The determiner 13 acquires the amounts of change in the individual biological indices output by the calculator 12 and reads out the thresholds of the individual biological indices stored in the storage unit 15. The determiner 13 compares the relative magnitudes of the amounts of change in the individual biological indices and the thresholds of the individual biological indices to determine a factor for stress. For example, in a case where the amount of change in each biological index is a positive value, the determiner 13 compares the relative magnitudes of the amount of change in the biological index and the positive threshold. In a case where the amount of change in each biological index is a negative value, the determiner 13 compares the relative magnitudes of the absolute value of the amount of change in the biological index and the absolute value of the negative threshold. Hereinafter, a more detailed description will be given by using the amount-of-change graph 120 and a determination table 130.

As illustrated in the amount-of-change graph 120, in a period A1, the absolute value of the amount of change in RRI is larger than the absolute value of the negative threshold 1b and the amount of change in CvRR is larger than the positive threshold 2a. Thus, the determiner 13 determines that a factor for stress felt by the measurement subject in the period A1 is an interpersonal-related factor. In a period B1, the amount of change in RRI is larger than the positive threshold 1a and the absolute value of the amount of change in CvRR is smaller than the absolute value of the negative threshold 2b. Thus, the determiner 13 determines that a factor for stress felt by the measurement subject in the period B1 is pain. In a period C1, the absolute value of the amount of change in RRI is smaller than the absolute value of the negative threshold 1b and the absolute value of the amount of change in CvRR is larger than the absolute value of the negative threshold 2b. Thus, the determiner 13 determines that a factor for stress felt by the measurement subject in the period C1 is thinking-induced fatigue.

In the determination table 130, the orientations and the number of arrows indicate a shift of the amount of change in each biological index based on the reference value (zero amount of change). A lateral arrow indicates that the amount of change in the biological index does not involve a change of exceeding the threshold.

Furthermore, the determiner 13 determines an intensity of the stress in accordance with the difference between the absolute value of the amount of change in RRI and the absolute value of the first threshold and the difference between the absolute value of the amount of change in CvRR and the absolute value of the second threshold.

The determiner 13 outputs information based on these determination results to the presenter 14. The presenter 14 is, for example, a display of a smartphone or tablet terminal. In addition, the determiner 13 stores the information based on the determination results in the storage unit 15. Accordingly, the measurement subject is able to retrieve the information based on the determination results at desired timing. At this time, the determiner 13 causes the presenter 14 to present the information based on the determination results in response to an operation of the measurement subject input by the input unit 16, such as a touch screen. For example, when the measurement subject inputs an instruction to retrieve necessary information by using the input unit 16 of the evaluation terminal 20, the determiner 13 causes the presenter 14 to present presentation information 140 in response to the instruction of the measurement subject. The presentation information 140 includes a time when the measurement subject felt stress, a factor for the stress, and measures for reducing the stress. The measures for reducing the stress include, for example, a message of suggesting a stress relief method or stress avoidance method that is based on the factor for the stress. The message may be, for example, “take a little break” or “do some stretches” in a case where the factor for the stress is thinking-induced fatigue, and may be “meditate for a while” or “take a deep breath” in a case where the factor for the stress is an interpersonal-related factor.

As described above, according to the first embodiment, a factor for stress of a measurement subject can be easily and accurately determined in the subject's daily life. Thus, the measurement subject is able to grasp his/her stress state and appropriate measures for reducing the stress more accurately than before. Accordingly, the measurement subject is able to appropriately and efficiently control his/her stress and is thus able to continue controlling stress.

Second Underlying Knowledge Forming Basis of the Present Disclosure

The inventors earnestly conducted a study in view of the foregoing issues described in “First Underlying Knowledge Forming Basis of the Present Disclosure”. The details of the study are as follows.

The inventors conducted the following monitoring tests to determine the relationship between factors for stress and biological indices acquired from biological information, such as heartbeat information and perspiration information.

Monitoring Tests

Four tasks related to different factors for stress were given to each of twenty subjects, and biological signals of the subjects performing the tasks were measured.

As the subjects twenty people who were male or female working adults or university students in their twenties to thirties and who did not show abnormal values in the results of questionnaires about their health and mental states were selected.

The four tasks were [1] a task related to stress from an interpersonal relationship, [2] a task related to stress from pain, [3] a task related to stress from thinking-induced fatigue 1, and [4] a task related to stress from thinking-induced fatigue 2. These tasks were carried out by each subject. The details of each task are similar to those in the monitoring tests described in “First Underlying Knowledge Forming Basis of the Present Disclosure”, and thus the description thereof is not given here.

The above monitoring tests were conducted on the individual subjects at the same time on different days in consideration of diurnal variation.

At-rest biological signals of the subject were biological signals measured for 5 minutes at the same position as that for performing a task before execution of each of the tasks [1] to [4]. Biological indices were calculated from the biological signals and set as reference values for calculating the amounts of change in biological indices. The amounts of change in biological indices are biological indices calculated from the biological signals of the subject measured during a task relative to the at-rest biological indices of the subject.

The measured biological signals were an electrocardiogram (ECG), breathing interval, fingertip skin temperature (SKT), and fingertip skin conductance (SC). These biological signals were measured simultaneously. A plurality of types of biological indices were acquired from each biological signal.

The method for calculating a biological index varies among biological indices. For example, in a case where the biological index is SKT, the SKT can be acquired by averaging fingertip skin temperatures in a certain section. The methods for calculating CvRR, HF, and LF are as those described above and thus the description thereof is not given here.

Subsequently, a combination of the amounts of change in biological indices having high performance to determine a factor for stress was considered. Specifically, linear discriminant analysis was performed by using the calculated amounts of change in RRI, CvRR, LF, HF, SC, and SKT. As a result of performing linear discriminant analysis using the amounts of change in all the biological indices, the determination accuracy was about 81.3%. In discrimination using a decision tree that is simpler, the determination accuracy was 77.5%.

In addition, as a result of performing linear discriminant analysis using the amounts of change in RRI, CvRR, and SC, the determination accuracy was 81.3%. In discrimination using a decision tree, the determination accuracy was 66.3%. Accordingly, it was found that relatively high determination accuracy is kept even when the amounts of change in three biological indices are used to determine a factor for stress.

On the other hand, as a result of performing linear discriminant analysis using the amounts of change in CvRR and SC, eliminating RRI as a biological index of heart rate, for example, the determination accuracy was 62.5%. Accordingly, it was found that the determination accuracy is significantly decreased by eliminating the amount of change in RRI as an index of heart rate from the amounts of change in biological indices to be used to determine a factor for stress.

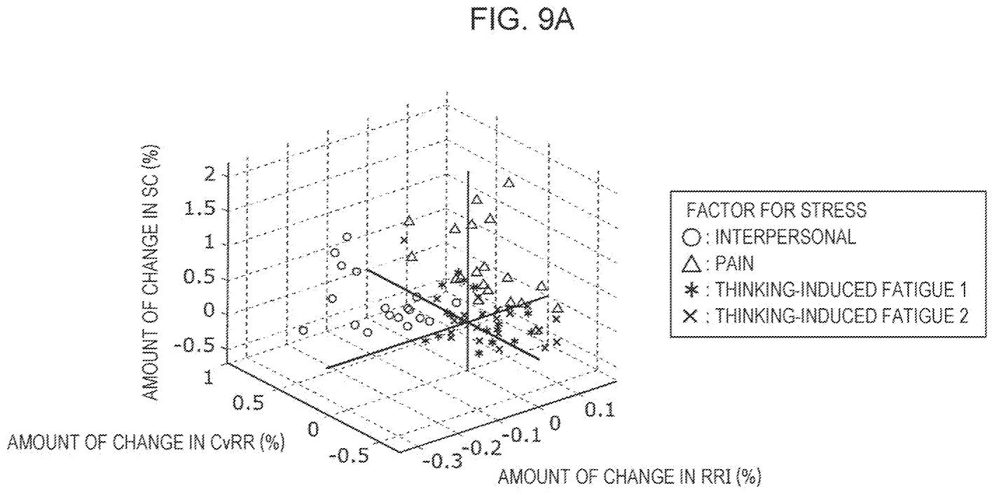

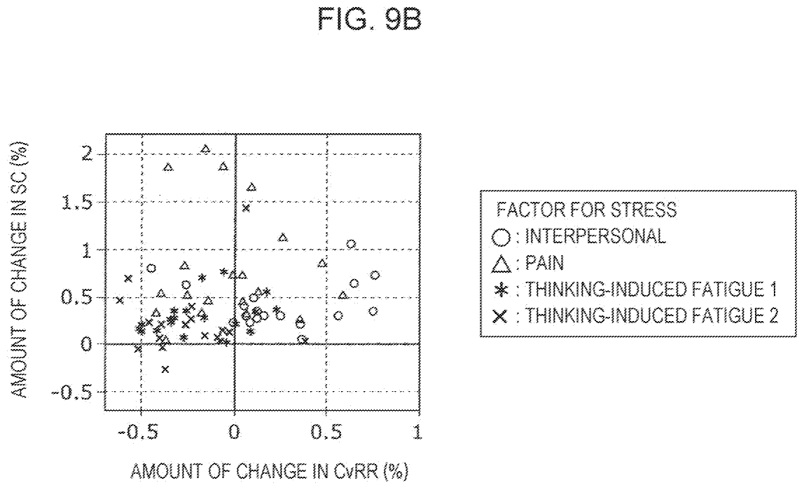

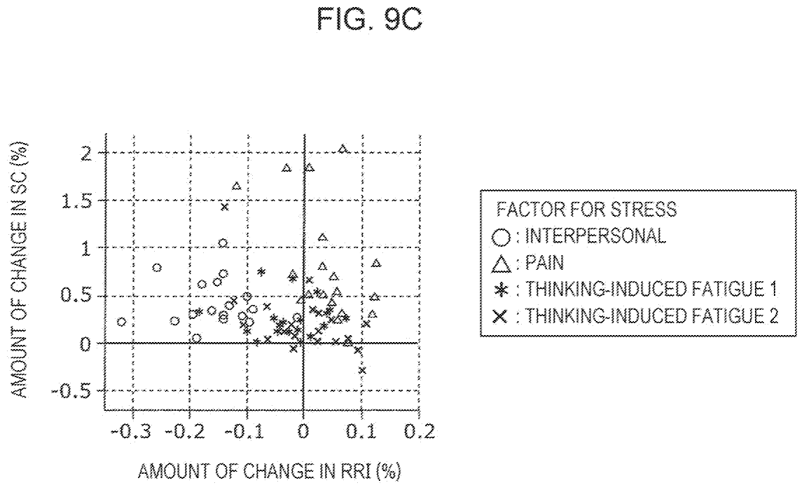

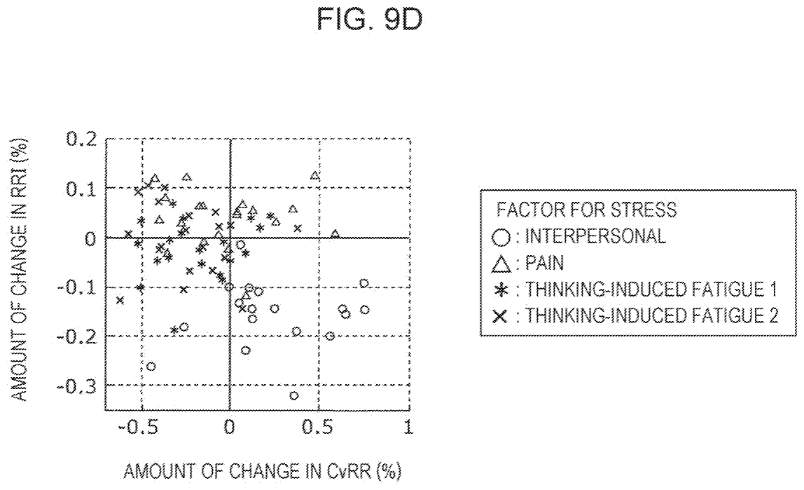

Therefore, factors for stress were determined by using the amount of change in RRI, the amount of change in CvRR, and the amount of change in SC as the amounts of change in biological indices. FIG. 9A is a graph that plots the amounts of change in the biological indices of the twenty subjects for individual factors for stress. FIG. 9B is a graph corresponding to FIG. 9A viewed from the positive side of the axis indicating the amount of change in RRI. FIG. 9C is a graph corresponding to FIG. 9A viewed from the negative side of the axis indicating the amount of change in CvRR. FIG. 9D is a graph corresponding to FIG. 9A viewed from the negative side of the axis indicating the amount of change in SC.

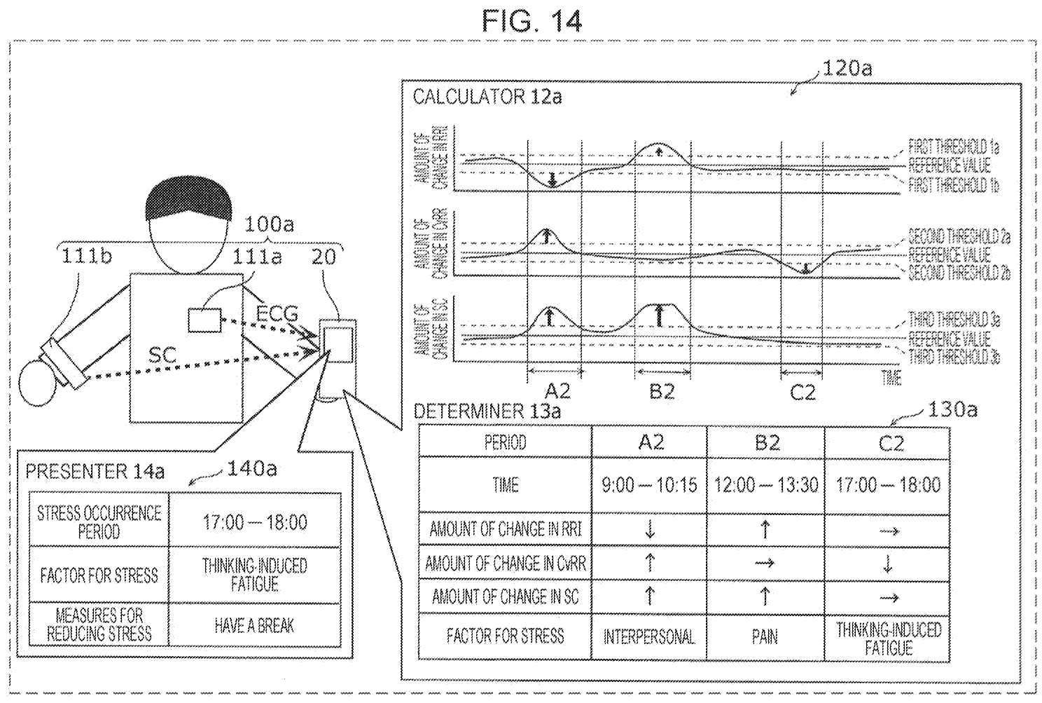

It was found from FIG. 9A to FIG. 9D that the trends in the amounts of change in the biological indices vary according to the type of the task that is performed. To make the trends in the changes clearer, average values of the amounts of change in the biological indices of the twenty subjects were calculated. FIG. 10A is a graph illustrating the average values of the amounts of change in the biological indices of the twenty subjects for the individual factors for stress plotted in FIG. 9A. FIG. 10B is a graph corresponding to FIG. 10A viewed from the positive side of the axis indicating the amount of change in RRI. FIG. 100 is a graph corresponding to FIG. 10A viewed from the negative side of the axis indicating the amount of change in CvRR. FIG. 10D is a graph corresponding to FIG. 10A viewed from the negative side of the axis indicating the amount of change in SC. It was found from FIG. 10A to FIG. 10D that the amounts of change in the biological indices have the following characteristic trends according to the factors for stress.

In a case where the factor for stress is an interpersonal-related factor, there is a trend that the amount of change in RRI significantly shifts to the negative side (i.e., the heart rate increases), the amount of change in CvRR shifts to the positive side, and the amount of change in SC shifts to the positive side. In a case where the factor for stress is pain, there is a trend that the amount of change in RRI shifts to the positive side (i.e., the heart rate decreases), the amount of change in CvRR slightly shifts to the negative side, and the amount of change in SC significantly shifts to the positive side. In a case where the factor for stress is thinking-induced fatigue, there is a trend that the amount of change in RRI very slightly shifts to the negative side (i.e., the heart rate hardly changes), the amount of change in CvRR significantly shifts to the negative side, and the amount of change in SC shifts to the positive side.

From the above results, it was found that the use of the amount of change in RRI, the amount of change in CvRR, and the amount of change in SC makes it possible to determine a factor for stress with relatively high accuracy. It was also found that the trends in these amounts of change vary according to a factor for stress. It was further found that a factor for stress of a subject can be easily and accurately determined on the basis of the trends in the amounts of change.

As a result of the above consideration, the inventors have acquired the knowledge that the amount of change in each biological index has a predetermined trend according to a factor for stress and particularly that a factor for stress can be determined with relatively high accuracy by using the amounts of change in biological indices related to (i) heart rate, (ii) heart rate variability, and (iii) skin conductance or skin temperature as indices for determination. In addition, on the basis of the result of the consideration, the inventors have conceived of a device that determines a factor for stress of a measurement subject by comparing the amounts of change in a plurality of types of biological indices acquired from the measurement subject with thresholds.

Accordingly, one embodiment of the present disclosure provides a stress evaluation device, a stress evaluation method, and a non-transitory computer-readable medium that are capable of determining a factor for stress of a measurement subject.

An overview of one aspect of the present disclosure is as follows.

A stress evaluation device according to one aspect of the present disclosure further includes a second sensor that measures at least one of a skin conductance or a skin temperature of the measurement subject. The calculator further calculates (iii) an amount of change in skin conductance or an amount of change in skin temperature. The amount of change in skin conductance is an amount of change from a reference value that is an at-rest skin conductance of the measurement subject to the skin conductance measured by the second sensor. The amount of change in skin temperature is an amount of change from a reference value that is an at-rest skin temperature of the measurement subject to the skin temperature measured by the second sensor. The determiner makes, in addition to the (I) and the (II), (III) a comparison between relative magnitudes of the amount of change in skin conductance or the amount of change in skin temperature and a third threshold to determine the factor for the stress of the measurement subject, and outputs information based on a determination result.

With the above configuration, the amounts of change in individual biological indices are calculated on the basis of at-rest biological indices of the measurement subject, and thus transition of the individual biological indices can be grasped more accurately. Thus, as a result of comparing the relative magnitudes of the amounts of change in individual biological indices and the thresholds of the individual biological indices, the factor for the stress can be determined.

For example, in the stress evaluation device according to the one aspect of the present disclosure, the amount of change in heart rate may be an amount of change to the heart rate measured at a first time, the amount of change in heart rate variability may be an amount of change to the heart rate variability measured at a second time, the amount of change in skin conductance or the amount of change in skin temperature may be an amount of change to the skin conductance or the skin temperature measured at a third time, the first threshold may be the heart rate measured at a certain time different from the first time, the second time, and the third time relative to the at-rest heart rate of the measurement subject, the second threshold may be the heart rate variability measured at the certain time relative to the at-rest heart rate variability of the measurement subject, and the third threshold may be the skin conductance measured at the certain time relative to the at-rest skin conductance of the measurement subject or the skin temperature measured at the certain time relative to the at-rest skin temperature of the measurement subject.

Here, the certain time is, for example, a time before the measurement subject feels stress. Accordingly, the first threshold, the second threshold, and the third threshold can be accurately set.

For example, in the case of comparing the relative magnitudes of the amounts of change in individual biological indices and the thresholds, biological indices measured at a predetermined time during sleep or immediately before bedtime of the measurement subject may be set as the thresholds of the individual biological indices. Accordingly, the thresholds can be set in consideration of menstrual variation of women, interannual variability, or the like without setting the certain time by the measurement subject, and thus the factor for the stress can be determined more accurately.

For example, in the stress evaluation device according to the one aspect of the present disclosure, the heart rate variability may be obtained by performing frequency analysis on heartbeat intervals of the measurement subject.

Accordingly, the stress evaluation device is capable of acquiring information about a breathing interval and a blood pressure from the frequency components of heart rate variability. Accordingly, the stress evaluation device is capable of using biological indices including detailed information of the measurement subject as indices as determination indices, and is thus capable of determining the factor for the stress of the measurement subject more accurately.

Accordingly, the stress evaluation device is capable of acquiring information about a breathing interval and a blood pressure from the frequency components of heart rate variability. Thus, the stress evaluation device is capable of using biological indices including detailed state of the measurement subject as indices (determination indices) for determining stress, and is thus capable of determining the factor for the stress of the measurement subject more accurately.

For example, in the stress evaluation device according to the one aspect of the present disclosure, in a case where the amount of change in heart rate is larger than the first threshold, the amount of change in heart rate variability is larger than the second threshold, and the amount of change in skin conductance or the amount of change in skin temperature is larger than the third threshold, the determiner may determine that the factor for the stress is an interpersonal-related factor.

With the above configuration, as a result of comparing the relative magnitudes of the amounts of change in individual biological indices and the thresholds of the individual biological indices, it can be determined that the factor for the stress is an interpersonal-related factor.

For example, in the stress evaluation device according to the one aspect of the present disclosure, in a case where the amount of change in heart rate is larger than the first threshold, the amount of change in heart rate variability is smaller than the second threshold, and the amount of change in skin conductance or the amount of change in skin temperature is larger than the third threshold, the determiner may determine that the factor for the stress is pain.

With the above configuration, as a result of comparing the relative magnitudes of the amounts of change in individual biological indices and the thresholds of the individual biological indices, it can be determined that the factor for the stress is pain.

For example, in the stress evaluation device according to the one aspect of the present disclosure, in a case where the amount of change in heart rate is smaller than the first threshold, the amount of change in heart rate variability is larger than the second threshold, and the amount of change in skin conductance or the amount of change in skin temperature is smaller than the third threshold, the determiner may determine that the factor for the stress is thinking-induced fatigue.

With the above configuration, as a result of comparing the relative magnitudes of the amounts of change in individual biological indices and the thresholds of the individual biological indices, it can be determined that the factor for the stress is thinking-induced fatigue.

For example, in the stress evaluation device according to the one aspect of the present disclosure, the determiner may further determine an intensity of the stress in accordance with a difference between the amount of change in heart rate and the first threshold, a difference between the amount of change in heart rate variability and the second threshold, and a difference between the amount of change in skin conductance or the amount of change in skin temperature and the third threshold, and may output a determination result as the information based on the determination result.

Accordingly, the measurement subject is able to know the intensity of his/her stress. Accordingly, the measurement subject is able to be aware of stress control more easily and grasp the trend in his/her stress more easily. For example, the measurement subject is able to recognize that the tolerable stress intensity varies among a plurality of types of factors for stress. Accordingly, the measurement subject becomes able to determine whether stress control is immediately necessary in accordance with the condition of stress. Thus, the measurement subject is able to efficiently control stress and is thus able to continuously control stress.

For example, the stress evaluation device according to the one aspect of the present disclosure may further include a presenter that presents the information based on the determination result output by the determiner. The information may include at least one selected from the group consisting of the factor for the stress, an intensity of the stress, and measures for reducing the stress.

Accordingly, the measurement subject is able to know his/her stress condition and a stress control method immediately after feeling stress, and is thus able to reduce accumulation of stress.

For example, in the stress evaluation device according to the one aspect of the present disclosure, the presenter may present the information by using a sound.

Accordingly, the measurement subject is able to easily know his/her stress condition and a stress control method in daily life, and is thus able to keep awareness about control of his/her stress more easily. Thus, the measurement subject is able to continuously control his/her stress.

For example, in the stress evaluation device according to the one aspect of the present disclosure, the presenter may present the information by using an image.

Accordingly, the measurement subject is able to visually grasp his/her stress condition and a stress control method, and is thus able to clearly be aware of control of his/her stress. Thus, the measurement subject is able to continuously control his/her stress.

In a stress evaluation method according to one aspect of the present disclosure, the acquiring includes acquiring at least one of a measured skin conductance or a measured skin temperature of the measurement subject, the calculating includes calculating (iii) an amount of change in skin conductance or an amount of change in skin temperature, the amount of change in skin conductance is an amount of change from a reference value that is an at-rest skin conductance of the measurement subject to the measured skin conductance, the amount of change in skin temperature is an amount of change from a reference value that is an at-rest skin temperature of the measurement subject to the measured skin temperature, and the determining includes making, in addition to the (I) and the (II), (III) a comparison between relative magnitudes of the amount of change in skin conductance or the amount of change in skin temperature and a third threshold to determine the factor for the stress of the measurement subject, and outputting information based on a determination result.

With the above method, the amounts of change in individual biological indices are calculated on the basis of at-rest biological indices of the measurement subject, and thus transition of the individual biological indices can be grasped more accurately. Thus, as a result of comparing the relative magnitudes of the amounts of change in individual biological indices and the thresholds of the individual biological indices, the factor for the stress can be determined.

It should be noted that general or specific embodiments may be implemented as a system, a device, a method, an integrated circuit, a computer program, a computer-readable recording medium such as a CD-ROM, or any selective combination thereof.

Hereinafter, a second embodiment of the present disclosure will be described in detail with reference to the attached drawings.

### Second Embodiment

Hereinafter, a stress evaluation device, a stress evaluation method, and a non-transitory computer-readable medium according to the second embodiment will be described by using specific examples.

Overview of Stress Evaluation Device

FIG. 11 is a diagram illustrating a schematic configuration of a stress evaluation device 100a according to the second embodiment. As illustrated in FIG. 11, the stress evaluation device 100a includes the first sensor 11a, a second sensor 11b, a calculator 12a, a determiner 13a, a presenter 14a, and a storage unit 15a. In the stress evaluation device 100a, for example, the first sensor 11a and the second sensor 11b include the first biological sensor 111a and a second biological sensor 111b (see FIG. 12), respectively, that are wearable and measures a biological signal of a measurement subject. The first sensor 11a calculates a plurality of types of biological indices from the biological signal measured by the first biological sensor 111a and outputs the calculated biological indices as measured biological indices to the calculator 12a. The second sensor 11b calculates at least one type of biological index from the biological signal measured by the second biological sensor 111b and outputs the calculated biological index as a measured biological index to the calculator 12a. The calculator 12a calculates average values of the individual biological indices of the measurement subject at rest (hereinafter also referred to as reference values) and thresholds of the individual biological indices, and stores the calculated average values and thresholds in the storage unit 15a. The calculator 12a also calculates average values of the measured biological indices and the amounts of change in the individual biological indices and outputs the calculated average values and amounts to the determiner 13a. The determiner 13a determines a factor for stress of the measurement subject in accordance with the amounts of change in the individual biological indices. More specifically, the determiner 13a compares the relative magnitudes of the amounts of change in the individual biological indices and the thresholds of the individual biological indices to determine a factor for stress. In addition, the determiner 13a determines an intensity of the stress in accordance with the differences between the amounts of change in the individual biological indices and the thresholds of the individual biological indices. Subsequently, the determiner 13a outputs information based on these determination results to the presenter 14a. At this time, the determiner 13a stores the information based on the determination results in the storage unit 15a. The presenter 14a presents the information based on the determination results. Furthermore, the stress evaluation device 100a may include an input unit 16a (see FIG. 12) for inputting an instruction of the measurement subject (user). In response to an instruction of the measurement subject input to the input unit 16a, the determiner 13a causes the presenter 14a to present the information about the determination results.

Configuration of Stress Evaluation Device

The configuration of the stress evaluation device 100a according to the second embodiment will be described in more detail. FIG. 12 is a diagram illustrating a specific example of the configuration of the stress evaluation device 100a based on the configuration illustrated in FIG. 11.

As illustrated in FIG. 12, the stress evaluation device 100a includes the first sensor 11a including the first biological sensor 111a and the first signal processor 112a, the second sensor 11b including the second biological sensor 111b and a second signal processor 112b, the calculator 12a, the determiner 13a, the presenter 14a, the storage unit 15a, and the input unit 16a.

The first biological sensor 111a and the second biological sensor 111b measure a biological signal of a measurement subject. The biological signal is a signal of biological information. The biological information is, for example, physiological information affected by stress, such as heartbeats, pulses, the number of breaths, blood oxygen saturation, blood pressure, or body temperature. For easy measurement, the biological information is heartbeat information, for example. The heartbeat information is information acquired from heartbeats. Alternatively, the biological information may be pulse wave information.

Each of the first biological sensor 111a and the second biological sensor 111b (hereinafter simply referred to as a “biological sensor”) is a sensor for biological information. For example, in a case where the biological sensor is a sensor that acquires heartbeat information (a heartbeat sensor), the heartbeat sensor is, for example, a sensor including a pair of detection electrodes that are to be in contact with a body surface of a measurement subject. The heartbeat information acquired by the heartbeat sensor is an electric signal acquired from heartbeats and is, for example, an ECG. The heartbeat sensor may include conductive adhesive gel electrodes or dry electrodes formed of conductive fibers. The heartbeat sensor is to be worn on the chest and has, for example, a wearable shape in which wear and electrodes are integrated together.

In a case where the biological sensor is a sensor that acquires pulse wave information (hereinafter a pulse wave sensor), the pulse wave sensor is, for example, a sensor that measures, with a phototransistor and a photodiode, a change in the amount of blood in blood vessels by using reflected light or transmitted light. The pulse wave sensor measures pulse wave information while being worn around a wrist of a user. The pulse wave sensor may be worn around an ankle, a finger, an upper arm, or the like. The shape of the pulse wave sensor is not limited to a band shape (for example, a wristwatch shape) and may be an attachable shape to be attached to the neck or the like, or an eyeglass shape. Alternatively, the pulse wave sensor may be an image sensor that measures pulse wave information by using a change in chromaticity of skin of the face or a hand and calculates pulses.

In a case where the biological information is the number of breaths, the biological sensor is, for example, a belt-shaped sensor that is to be worn around the chest or abdomen and that includes a pressure sensor, or a temperature sensor to be worn under the nose.

In a case where the biological information is blood oxygen saturation, the biological sensor is, for example, a sensor that measures, with a phototransistor and two types of photodiodes, a change in saturated oxygen concentration in blood in blood vessels by using reflected light or transmitted light.

In a case where the biological information is blood pressure, the biological sensor is, for example, a belt-shaped sensor that is to be worn around an upper arm and a fingertip or a radial bone and that includes a pressure sensor.

In a case where the biological information is body temperature, the biological sensor is, for example, a thermocouple sensor to be attached to a portion where capillary contraction is likely to occur due to stress, such as a palm or the tip of the nose.

In a case where the biological information is perspiration, the biological sensor is, for example, a sensor including a pair of detection electrodes that are to be in contact with a portion where perspiration is likely to occur due to stress, such as a palm or the face.

The biological signals measured by the first biological sensor 111a and the second biological sensor 111b are output to the first signal processor 112a and the second signal processor 112b, respectively.

The first signal processor 112a calculates a plurality of types of biological indices from the one biological signal measured by the first biological sensor 111a. In the second embodiment, the first biological sensor 111a is a heartbeat sensor. As described above, in a case where the biological signal of heartbeats is an ECG, the plurality of types of biological indices are RRI, CvRR, HF, LF, and the like. The RRI is an index of heart rate, whereas the CvRR, HF, and LF are indices of heart rate variability. Furthermore, the first signal processor 112a may calculate biological indices of variations in the number of breaths and blood pressure from the frequency components of heart rate variability. Among the plurality of types of biological indices, a combination of RRI and CvRR is a combination that achieves relatively high determination accuracy. Thus, in the second embodiment, a description will be given of an example in which a first biological index and a second biological index are RRI and CvRR, respectively. The methods for calculating RRI and CvRR are as described above regarding the monitoring tests. The first signal processor 112a outputs the calculated first biological index and second biological index to the calculator 12a.

The second signal processor 112b calculates at least one type of biological index from the one biological signal measured by the second biological sensor 111b. In the second embodiment, a third biological index is calculated. As described above, in a case where the biological information is perspiration, the second biological sensor 111b a sensor including a pair of detection electrodes. In a case where the biological information is body temperature, the second biological sensor 111b is, for example, a thermocouple sensor. The second biological sensor 111b is worn, for example, around a finger of a measurement subject. In a case where the biological information is perspiration, the second signal processor 112b calculates a skin conductance. In a case where the biological information output from the second biological sensor 111b is body temperature, the second signal processor 112b calculates a skin temperature. Thus, in the second embodiment, the third biological index is skin conductance or skin temperature. The second signal processor 112b outputs the calculated third biological index to the calculator 12a.

The calculator 12a acquires the first biological index and second biological index output by the first signal processor 112a and calculates, from the acquired first biological index and second biological index, the amount of change in the first biological index and the amount of change in the second biological index. Also, the calculator 12a acquires the third biological index output by the second signal processor 112b and calculates, from the acquired third biological index, the amount of change in the third biological index. The amount of change in a biological index is a measured biological index relative to an at-rest biological index of a measurement subject (hereinafter also referred to as a reference value), and is expressed as a difference or a ratio. The reference values of the individual biological indices are stored in the storage unit 15a. The calculator 12a reads out the reference values of the individual biological indices stored in the storage unit 15a and calculates the amounts of change in the individual biological indices relative to the reference values. The calculator 12a outputs the calculated amounts of change in the individual biological indices to the determiner 13a. The reference values may vary according to a season or a menstrual cycle of the measurement subject and thus may be updated at predetermined intervals.

The calculator 12a also calculates the thresholds of the individual biological indices. For example, in a case where the first biological index is heart rate, the amount of change in heart rate is the amount of change to a heart rate measured at a first time. A first threshold is a threshold of the first biological index and is, for example, a threshold of RRI, which is an index of heart rate. The first threshold is a heart rate measured at a certain time relative to an at-rest heart rate of the measurement subject. For example, in a case where the second biological index is heart rate variability, the amount of change in heart rate variability is the amount of change to a heart rate variability measured at a second time. A second threshold is a threshold of the second biological index and is, for example, a threshold of CvRR, which is an index of heart rate variability. The second threshold is a heart rate variability measured at the certain time relative to an at-rest heart rate variability of the measurement subject. For example, in a case where the third biological index is skin conductance or skin temperature, the amount of change in skin conductance or skin temperature is the amount of change to a skin conductance or a skin temperature measured at a third time. A third threshold is a threshold of the third biological index and is, for example, a threshold of skin conductance or a threshold of skin temperature. The third threshold is a skin conductance measured at the certain time relative to an at-rest skin conductance of the measurement subject or a skin temperature measured at the certain time relative to an at-rest skin temperature of the measurement subject. Each of these thresholds is the amount of change in the biological index, which is a difference or a ratio between the reference value and the measurement value of the biological index measured at the certain time that is different from the first time, the second time, and the third time. Here, the certain time is, for example, a time before the measurement subject feels stress.

In the second embodiment, a description will be given of a case where the first time, the second time, and the third time are the same. Alternatively, the first time, the second time, and the third time may be different from each other. For example, the first signal processor 112a may calculate a plurality of types of heart rate and heart rate variability in a time-division manner from the one biological signal measured by the first biological sensor 111a. At this time, the calculator 12a calculates the amount of change to the heart rate measured at the first time and calculates the amount of change to the heart rate variability measured at the second time different from the first time. In addition, the second signal processor 112b may calculate a skin conductance or a skin temperature from the one biological signal measured by the second biological sensor 111b. At this time, the calculator 12a calculates the amount of change to the skin conductance or the skin temperature measured at the third time. The third time may be the same as the first time or the second time.

The calculator 12a reads out the thresholds of the individual biological indices stored in the storage unit 15a and compares the relative magnitudes of the amounts of change in the individual biological indices and the thresholds of the individual biological indices. Subsequently, the calculator 12a determines a period during which at least one of the amounts of change in the individual biological indices exceeds the threshold for a predetermined time to be a stress occurrence period. The stress occurrence period is a period during which the measurement subject feels stress. The calculator 12a calculates representative values of the amounts of change in the individual biological indices from the amounts of change in the individual biological indices during the stress occurrence period. As the representative values of the amounts of change in the individual biological indices during the stress occurrence period, for example, average values of the amounts of change in the individual biological indices during the stress occurrence period may be used, or values having the largest differences from the reference values (maximum values) may be used.

The determiner 13a acquires the representative values of the amounts of change in the individual biological indices output by the calculator 12a and reads out the first, second, and third thresholds stored in the storage unit 15a. The determiner 13a compares the relative magnitudes of the representative value of the amount of change in the first biological index and the first threshold, compares the relative magnitudes of the representative value of the amount of change in the second biological index and the second threshold, and compares the relative magnitudes of the representative value of the amount of change in the third biological index and the third threshold, thereby determining a factor for stress of the measurement subject. In other words, the determiner 13a determines a factor for stress in each stress occurrence period. The representative value of the amount of change in a biological index is an example of the amount of change in the biological index, and thus hereinafter the representative value of the amount of change in a biological index will also be referred to as the amount of change in the biological index.

Specifically, in a case where the amount of change in the first biological index (here, heart rate) is larger than the first threshold, the amount of change in the second biological index (here, heart rate variability) is larger than the second threshold, and the amount of change in the third biological index (here, skin conductance or skin temperature) is larger than the third threshold, the determiner 13a determines that a factor for stress is an interpersonal-related factor. In a case where the amount of change in the first biological index is larger than the first threshold, the amount of change in the second biological index is smaller than the second threshold, and the amount of change in the third biological index is larger than the third threshold, the determiner 13a determines that a factor for stress is pain. In a case where the amount of change in the first biological index is smaller than the first threshold, the amount of change in the second biological index is larger than the second threshold, and the amount of change in the third biological index is smaller than the third threshold, the determiner 13a determines that a factor for stress is thinking-induced fatigue.

Furthermore, the determiner 13a determines an intensity of the stress in accordance with the difference between the amount of change in the first biological index and the first threshold, the difference between the amount of change in the second biological index and the second threshold, and the difference between the amount of change in the third biological index and the third threshold, and outputs a determination result as information based on the determination result. The information based on the determination result includes, for example, at least one of the factor for the stress, the intensity of the stress, or measures for reducing the stress. The measures for reducing the stress may be, for example, a stress relief method or a stress avoidance method. The measures for reducing the stress are included in a presentation information table, which will be described below. The determiner 13a reads out appropriate measures for reducing the stress from the presentation information table stored in the storage unit 15a and outputs the measures to the presenter 14a.

In addition, the determiner 13a stores the information based on the determination result in the storage unit 15a. At this time, the determiner 13a may store the information based on the determination result in the storage unit 15a in association with information about the time when the measurement subject felt the stress.

The presenter 14a presents the information based on the determination result output by the determiner 13a. The presenter 14a may present the information based on the determination result by using a sound or an image. In a case where the presenter 14a presents the information by using a sound, the presenter 14a is, for example, a speaker. In a case where the presenter 14a presents the information by using an image, the presenter 14a is, for example, a display.

The storage unit 15a stores the reference values of the individual biological indices, the thresholds of the individual biological indices, the presentation information table, and the like. The presentation information table is a table of presentation information, such as measures for reducing stress, presented in accordance with a factor for stress and an intensity of the stress. As described above, the reference values and thresholds of the individual biological indices may be updated at predetermined intervals. Also, the presentation information table may be updated at predetermined intervals.

The storage unit 15a also stores the information based on the determination result output by the determiner 13a, such as the factor for the stress, the intensity of the stress, and the measures for reducing the stress. At this time, the storage unit 15a may store the information based on the determination result in association with the stress occurrence period. Accordingly, the measurement subject is able to retrieve the information based on the determination result at desired timing. At this time, the determiner 13a causes the presenter 14a to present the information based on the determination result in response to an operation of the measurement subject input by the input unit 16a.

The input unit 16a outputs an operation signal indicating the operation performed by the measurement subject to the determiner 13a. The input unit 16a is, for example, a keyboard, a mouse, a touch screen, a microphone, or the like. The operation signal is a signal for setting a method for extracting the information based on the determination result or a presentation method in the presenter 14a. On the basis of the setting input to the input unit 16a, the presenter 14a presents various forms of determination results, for example, a change in stress during a predetermined period, a factor for stress to which the measurement subject is susceptible, and measures for reducing stress suitable for the measurement subject. Accordingly, the measurement subject is able to grasp middle-term and long-term trends in stress as well as a short-term trend in stress. In this way, the measurement subject is able to know effective measures for reducing stress suitable for the measurement subject and is thus able to control the stress in a middle-term or long-term vision.

Stress Evaluation Method

The stress evaluation method according to the second embodiment will be described in detail with reference to FIG. 13. FIG. 13 is a flowchart for describing the stress evaluation method according to the second embodiment.

The stress evaluation method according to the second embodiment includes an acquisition step S100 of acquiring (i) a measured heart rate, (ii) a measured heart rate variability, and (iii) a measured skin conductance or skin temperature of a measurement subject; a calculation step S200 of calculating (i) the amount of change in heart rate, (ii) the amount of change in heart rate variability, and (iii) the amount of change in skin conductance or the amount of change in skin temperature; and a determination step S300 of determining a factor for stress of the measurement subject in accordance with (i) the amount of change in heart rate, (ii) the amount of change in heart rate variability, and (iii) at least one of the amount of change in skin conductance or the amount of change in skin temperature and outputting information based on a determination result. The amount of change in heart rate is the amount of change from a reference value that is an at-rest heart rate of the measurement subject to the heart rate measured by the first sensor 11a. The amount of change in heart rate variability is the amount of change from a reference value that is an at-rest heart rate variability of the measurement subject to the heart rate variability measured by the first sensor 11a. The amount of change in skin conductance is the amount of change from a reference value that is an at-rest skin conductance of the measurement subject to the skin conductance measured by the second sensor 11b. The amount of change in skin temperature is the amount of change from a reference value that is an at-rest skin temperature of the measurement subject to the skin temperature measured by the second sensor 11b. In the determination step S300, (I) a comparison between relative magnitudes of the amount of change in heart rate and the first threshold, (II) a comparison between relative magnitudes of the amount of change in heart rate variability and the second threshold, and (III) a comparison between relative magnitudes of the amount of change in skin conductance or the amount of change in skin temperature and the third threshold are made to determine the factor for the stress. In the second embodiment, the stress evaluation method further includes a presentation step S400 of presenting the information based on the determination result acquired in the determination step S300.

Hereinafter, the individual steps will be described in more detail.

In the acquisition step S100, the calculator 12a acquires a plurality of types of biological indices of the measurement subject measured by the first sensor 11a and the second sensor 11b. In the first sensor 11a, the first biological sensor 111a measures heartbeat information (here, an ECG), and the first signal processor 112a calculates an index of heart rate and an index of heart rate variability. In the second sensor 11b, the second biological sensor 111b measures biological information of temperature or perspiration, and the second signal processor 112b calculates a skin temperature (SKT) or a skin conductance (SC). As described above, the biological information may be physiological information affected by stress, such as heartbeats, pulses, the number of breaths, blood oxygen saturation, blood pressure, body temperature, or perspiration. In particular, heartbeat information can be measured in real time more easily and with a smaller burden on the measurement subject than other biological information, such as pulses, the number of breaths, blood pressure, and blood oxygen saturation, when a wearable biological sensor is used. Thus, as a result of using heartbeat information of the measurement subject as biological information, the stress state of the measurement subject can be appropriately evaluated.

The biological indices acquired from the heartbeat information include RRI which is an index of heart rate, and CvRR, LF, HF, and LF/HF which are indices of heart rate variability. In this way, a plurality of types of biological indices can be acquired from one piece of biological information. As described above, a combination of these biological indices makes it possible to determine a factor for stress with relatively high determination accuracy, and thus reliable evaluation can be acquired.

Referring back to FIG. 6, the heartbeat information is, for example, an ECG, and is the ECG waveform illustrated in FIG. 6. The ECG waveform is formed of a P wave that reflects electrical excitation of the cardiac atria; a Q wave, an R wave, and an S wave that reflect electrical excitation of the cardiac ventricles; and a T wave that reflects repolarization of excited cardiac myocytes of the ventricles. Among these waves, the R wave has the largest wave height (potential difference) and is the most robust against noise such as a myoelectric potential. Thus, the interval between the peaks of the R waves of two consecutive heartbeats in the ECG waveform, that is, the heartbeat interval (RRI), is calculated. A heart rate is calculated by multiplying the reciprocal of RRI by 60.

Furthermore, as described above regarding the monitoring tests in “First Underlying Knowledge Forming Basis of the Present Disclosure”, CvRR is calculated from RRIs by normalizing a standard deviation SD of the RRIs in a certain time period by using an average value of the RRIs in the certain time period, by using the above equation (1).

The first signal processor 112a detects, from the heartbeat information acquired by the first biological sensor 111a, an electric signal (R waves) generated when the left ventricle suddenly contracts to send blood from the heart, and calculates an RRI. To detect R waves, for example, an existing method such as the Pan & Tompkins method is used.

Next, a description will be given of a method for calculating the amount of variation in the heartbeat intervals (RRIs) from the detected R waves by the calculator 12a.

Referring back to FIG. 7, the first signal processor 112a calculates the amount of variation in the RRIs from acquired detection data of R waves in the following manner.

As illustrated in FIG. 7(a), the first signal processor 112a calculates RRIs, each being an interval between the peaks of R waves of two consecutive heartbeats. The first signal processor 112a converts the calculated RRIs into the relationship between two axes, time and RRI. The converted data is discrete data of irregular intervals, and thus the calculator 12a converts the RRI chronological data into the regular-interval chronological data illustrated in FIG. 7(b). Next, the calculator 12a performs frequency analysis on the regular-interval chronological data by using fast Fourier transform (FFT), thereby acquiring frequency components of heart rate variability illustrated in FIG. 7(c).

The frequency components of heart rate variability can be divided into, for example, a high-frequency component HF and a low-frequency component LF. As described above regarding the monitoring tests, the HF is considered to reflect the amount of parasympathetic nerve activity, and the LF is considered to reflect the amount of sympathetic nerve activity and parasympathetic nerve activity. Thus, LF/HF, which is the ratio of LF to HF, is considered to indicate the amount of sympathetic nerve activity.

In this way, the first sensor 11a calculates a plurality of types of biological indices from heartbeat information.

As described above, in the acquisition step S100, the calculator 12a acquires two types of biological indices (here, heart rate and heart rate variability) output from the first sensor 11a and one type of biological index (here, skin conductance) output from the second sensor 11b.

Subsequently, in the calculation step S200, the calculator 12a calculates the amounts of change in the individual biological indices acquired in the acquisition step S100. As described above, the amount of change in each biological index is acquired by, for example, calculating the ratio or difference between the acquired value of the biological index and the reference value of the biological index, which is the value of the at-rest biological index of the measurement subject. The calculator 12a reads out and uses the reference values of the individual biological indices stored in the storage unit 15a.

In a case where the amount of change in each biological index is expressed as a difference, the amount of change is calculated by subtracting the reference value of the biological index from the value of the biological index acquired in the acquisition step S100. For example, the amount of change in heart rate is calculated by subtracting the reference value of heart rate from the value of heart rate of the measurement subject acquired in the acquisition step S100. In a case where the amount of change in each biological index is expressed as a ratio, the amount of change is calculated by dividing the value of the biological index acquired in the acquisition step S100 by the reference value of the biological index. For example, the amount of change in heart rate is calculated by dividing the value of heart rate of the measurement subject acquired in the acquisition step S100 by the reference value of heart rate.

In the above-described manner, the calculator 12a calculates the amounts of change in the individual biological indices in the calculation step S200.

Subsequently, in the determination step S300, the determiner 13a determines a factor for stress in accordance with the amounts of change in the individual biological indices calculated in the calculation step S200. The determiner 13a compares the relative magnitudes of the amounts of change in the individual biological indices and the thresholds of the individual biological indices to determine a factor for stress of the measurement subject. Specifically, in the determination step S300, in a case where the amount of change in heart rate is larger than the first threshold, the amount of change in heart rate variability is larger than the second threshold, and the amount of change in skin conductance or the amount of change in skin temperature is larger than the third threshold, the determiner 13a determines that a factor for stress is an interpersonal-related factor. In a case where the amount of change in heart rate is larger than the first threshold, the amount of change in heart rate variability is smaller than the second threshold, and the amount of change in skin conductance or the amount of change in skin temperature is larger than the third threshold, the determiner 13a determines that a factor for stress is pain. In a case where the amount of change in heart rate is smaller than the first threshold, the amount of change in heart rate variability is larger than the second threshold, and the amount of change in skin conductance or the amount of change in skin temperature is smaller than the third threshold, the determiner 13a determines that a factor for stress is thinking-induced fatigue.

Furthermore, the determiner 13a determines an intensity of the stress in accordance with the difference between the amount of change in heart rate and the first threshold, the difference between the amount of change in heart rate variability and the second threshold, and the difference between the amount of change in skin conductance or the amount of change in skin temperature and the third threshold, and outputs a determination result as information based on the determination result.

The first threshold is the threshold of heart rate and is a heart rate measured at the certain time relative to the at-rest heart rate of the measurement subject. The second threshold is the threshold of heart rate variability and is a heart rate variability measured at the certain time relative to the at-rest heart rate variability of the measurement subject. The third threshold is the threshold of skin conductance or skin temperature and is a skin conductance or a skin temperature measured at the certain time relative to the at-rest skin conductance or skin temperature of the measurement subject. These thresholds are calculated by the calculator 12a and stored in the storage unit 15a. The determiner 13a reads out and uses the thresholds of the individual biological indices stored in the storage unit 15a. As described above, the certain time is a time before the measurement subject feels stress.

As the threshold of each biological index, a threshold in a case where the amount of change in the biological index is a positive value and a threshold in a case where the amount of change in the biological index is a negative value are set. The reference value corresponds to zero amount of change. The relative magnitudes of the amount of change in each biological index and the threshold are compared in the following manner. In a case where the amount of change in the biological index is a positive value, the relative magnitudes of the amount of change in the biological index and the positive threshold are compared with each other. In a case where the amount of change in the biological index is a negative value, the relative magnitudes of the absolute value of the amount of change in the biological index and the absolute value of the negative threshold are compared with each other. The threshold of each biological index may be a fixed value, may be updated at predetermined intervals, or may be updated on the basis of daily measurements.

The threshold may be calculated by relatively simple machine learning, such as linear discrimination or a decision tree. Accordingly, a determination reference value and a threshold suitable for the measurement subject can be set, and thus a factor for stress can be determined more accurately.

As described above, in the determination step S300, the relative magnitudes of the amounts of change in the individual biological indices and the thresholds of the individual biological indices are compared with each other, and accordingly a factor for stress of the measurement subject is determined.

Subsequently, in the presentation step S400, the presenter 14a presents information based on the determination result obtained by the determiner 13a. The presenter 14a may present the information based on the determination result by using a sound or an image. The information based on the determination result includes at least one of a factor for stress, an intensity of stress, or measures for reducing stress. The presenter 14a displays various forms of determination results on the basis of the setting input by the measurement subject in the input unit 16a.

Example of Use of Stress Evaluation Device

An example of use of the stress evaluation device 100a according to the second embodiment will be described in detail. FIG. 14 is a diagram for describing an example of use of the stress evaluation device 100a according to the second embodiment.

As illustrated in FIG. 14, the stress evaluation device 100a includes the first biological sensor 111a, which is a part of the first sensor 11a, the second biological sensor 111b, which is a part of the second sensor 11b, and the evaluation terminal 20 including the components other than the first biological sensor 111a and the second biological sensor 111b. A measurement subject wears the first biological sensor 111a such that the first biological sensor 111a is in contact with the skin of the chest and measures an ECG. The first biological sensor 111a may include conductive adhesive gel electrodes or dry electrodes formed of conductive fibers. The first biological sensor 111a transmits a measured electric signal of heartbeats to the evaluation terminal 20 through communication.

The second biological sensor 111b is a wristwatch-shaped sensor and includes a sensor electrode to be used while being attached to a palm. The second biological sensor 111b measures, using the sensor electrode, a skin potential of the palm and transmits a measurement result to the evaluation terminal 20 through communication. Furthermore, the second biological sensor 111b may include a thermocouple sensor to be used while being attached to a fingertip. Accordingly, the second biological sensor 111b is capable of measuring a fingertip temperature by using the thermocouple sensor. The communication method between the first biological sensor 111a and the evaluation terminal 20 and between the second biological sensor 111b and the evaluation terminal 20 may be wireless communication using Bluetooth (registered trademark) or the like, or may be wired communication.

The evaluation terminal 20 includes the first signal processor 112a of the first sensor 11a, the second signal processor 112b of the second sensor 11b, the calculator 12a, the determiner 13a, the presenter 14a, the storage unit 15a, and the input unit 16a. The first signal processor 112a and the second signal processor 112b receive the biological signals transmitted by the first biological sensor 111a and the second biological sensor 111b through communication, respectively.

The first signal processor 112a calculates, from the received electric signal of heartbeats, RRI which is an index of heart rate and CvRR which is an index of heart rate variability, and outputs these biological indices to the calculator 12a. The second signal processor 112b calculates, from the received signal of skin potential, skin conductance (SC) which is an index of perspiration, and outputs the SC to the calculator 12a. In a case where the second biological sensor 111b measures a skin temperature, the second signal processor 112b receives a signal of the skin temperature from the second biological sensor 111b, calculates a skin temperature (SKT) which is an index of body temperature, and outputs the SKT to the calculator 12a.

The calculator 12a acquires the RRI and CvRR output by the first signal processor 112a and reads out the reference value of RRI and the reference value of CvRR stored in the storage unit 15a. The calculator 12a also acquires the SC output by the second signal processor 112b and reads out the reference value of SC stored in the storage unit 15a. The calculator 12a calculates the amounts of change in these biological indices relative to the read out reference values. The amount of change in each biological index is expressed as a difference or a ratio. In the second embodiment, the amount of change is expressed as a ratio.

As described above, the calculator 12a calculates the thresholds of the individual biological indices and outputs the thresholds to the storage unit 15a. As the threshold of each biological index, a threshold in a case where the amount of change in the biological index is a positive value and a threshold in a case where the amount of change in the biological index is a negative value are set. The reference value corresponds to zero amount of change. Specifically, in a case where the amount of change in each biological index is a positive value, the positive threshold is a value larger than the reference value, and corresponds to a first threshold 1a (hereinafter a positive threshold 1a), a second threshold 2a (hereinafter a positive threshold 2a), and a third threshold 3a (hereinafter a positive threshold 3a) in an amount-of-change graph 120a. In a case where the amount of change in each biological index is a negative value, the negative threshold is a value smaller than the reference value, and corresponds to a first threshold 1b (hereinafter a negative threshold 1b), a second threshold 2b (hereinafter a negative threshold 2b), and a third threshold 3b (hereinafter a negative threshold 3b) in the amount-of-change graph 120a. In addition, the calculator 12a calculates the reference values of the individual biological indices and outputs the reference values to the storage unit 15a. The reference value of each biological index corresponds to zero amount of change in the biological index. For example, in the amount-of-change graph 120a, the solid line between the positive threshold 1a and the negative threshold 1b corresponds to the reference value. The positive threshold and the negative threshold may or may not be set at regular intervals with the reference value (zero amount of change) interposed therebetween. These thresholds may be appropriately set in accordance with the amount of change in each biological index.

The determiner 13a acquires the amounts of change in the individual biological indices output by the calculator 12a and reads out the thresholds of the individual biological indices stored in the storage unit 15a. The determiner 13a compares the relative magnitudes of the amounts of change in the individual biological indices and the thresholds of the individual biological indices to determine a factor for stress. For example, in a case where the amount of change in each biological index is a positive value, the determiner 13a compares the relative magnitudes of the amount of change in the biological index and the positive threshold. In a case where the amount of change in each biological index is a negative value, the determiner 13a compares the relative magnitudes of the absolute value of the amount of change in the biological index and the absolute value of the negative threshold. Hereinafter, a more detailed description will be given by using the amount-of-change graph 120a and a determination table 130a.

As illustrated in the amount-of-change graph 120a, in a period A2, the absolute value of the amount of change in RRI is larger than the absolute value of the negative threshold 1b, the amount of change in CvRR is larger than the positive threshold 2a, and the amount of change in skin conductance is larger than the positive threshold 3a. Thus, the determiner 13a determines that a factor for stress felt by the measurement subject in the period A2 is an interpersonal-related factor. In a period B2, the amount of change in RRI is larger than the positive threshold 1a, the absolute value of the amount of change in CvRR is smaller than the absolute value of the negative threshold 2b, and the amount of change in skin conductance is larger than the positive threshold 3a. Thus, the determiner 13a determines that a factor for stress felt by the measurement subject in the period B2 is pain. In a period C2, the absolute value of the amount of change in RRI is smaller than the absolute value of the negative threshold 1b, the absolute value of the amount of change in CvRR is larger than the absolute value of the negative threshold 2b, and the absolute value of the amount of change in skin conductance is smaller than the absolute value of the negative threshold 3b. Thus, the determiner 13a determines that a factor for stress felt by the measurement subject in the period C2 is thinking-induced fatigue.

In the determination table 130a, the orientations and the number of arrows indicate a shift of the amount of change in each biological index based on the reference value (zero amount of change). A lateral arrow indicates that the amount of change in the biological index does not involve a change of exceeding the threshold.

Furthermore, the determiner 13a determines an intensity of the stress in accordance with the difference between the absolute value of the amount of change in RRI and the absolute value of the first threshold, the difference between the absolute value of the amount of change in CvRR and the absolute value of the second threshold, and the difference between the absolute value of the amount of change in SC and the absolute value of the third threshold.

The determiner 13a outputs information based on these determination results to the presenter 14a. The presenter 14a is, for example, a display of a smartphone or tablet terminal. In addition, the determiner 13a stores the information based on the determination results in the storage unit 15a. Accordingly, the measurement subject is able to retrieve the information based on the determination results at desired timing. At this time, the determiner 13a causes the presenter 14a to present the information based on the determination results in response to an operation of the measurement subject input by the input unit 16a, such as a touch screen. For example, when the measurement subject inputs an instruction to retrieve necessary information by using the input unit 16a of the evaluation terminal 20, the determiner 13a causes the presenter 14a to present presentation information 140a in response to the instruction of the measurement subject. The presentation information 140a includes a time when the measurement subject felt stress, a factor for the stress, and measures for reducing the stress. The measures for reducing the stress include, for example, a message of suggesting a stress relief method or stress avoidance method that is based on the factor for the stress. The message may be, for example, “take a little break” or “do some stretches” in a case where the factor for the stress is thinking-induced fatigue, and may be “meditate for a while” or “take a deep breath” in a case where the factor for the stress is an interpersonal-related factor.

As described above, according to the second embodiment, a factor for stress of a measurement subject can be easily and accurately determined in the subject's daily life. Thus, the measurement subject is able to grasp his/her stress state and appropriate measures for reducing the stress more accurately than before. Accordingly, the measurement subject is able to appropriately and efficiently control his/her stress and is thus able to continue controlling stress.

The stress evaluation devices, the stress evaluation methods, and the non-transitory computer-readable media according to the embodiments of the present disclosure have been described above. The present disclosure is not limited to these embodiments. An embodiment implemented by applying various modifications conceived of by a person skilled in the art to any one of the embodiments, or another embodiment implemented by combining some of the components in the embodiments, is also included in the scope of the present disclosure without deviating from the gist of the present disclosure.

In the above embodiments, an example has been given in which heartbeat information is used as biological information and an index of heart rate and an index of heart rate variability are used as a plurality of types of biological indices acquired from the heartbeat information. The present disclosure is not limited thereto. For example, an entropy E representing the degree of autonomic nerve activity, and a tone T representing autonomic nerve balance, may be used. In the above embodiments, an example has been given in which RRI is used as an index of heart rate and CvRR, LF, and HF are used as indices of heart rate variability. Alternatively, an index indicating heart rate variability other than these indices may be used.

In the first embodiment, an example has been given in which the stress evaluation device 100 includes the first biological sensor 111a and the evaluation terminal 20. Alternatively, for example, the stress evaluation device 100 may include the first sensor 11a and an evaluation terminal including the components other than the first sensor 11a.

In the second embodiment, an example has been given in which the stress evaluation device 100a includes the first biological sensor 111a, the second biological sensor 111b, and the evaluation terminal 20. Alternatively, for example, the stress evaluation device 100a may include the first sensor 11a, the second sensor 11b, and an evaluation terminal including the components other than the first sensor 11a and the second sensor 11b.

The stress evaluation device may be an integral device in which all the components are integrated in one device. In the above embodiments, an example has been given in which a heartbeat sensor is used as a biological sensor. Alternatively, a pulse wave sensor may be used as a biological sensor. In this case, the stress evaluation device may be a wristwatch-shaped device including a display.

In the first embodiment, an example has been given in which the evaluation terminal 20 is a smartphone or a tablet terminal. The smartphone or the tablet terminal may include the presenter 14 and the input unit 16, and the first signal processor 112a, the calculator 12, the determiner 13, and the storage unit 15 may be provided in a server connected through a communication network such as the Internet.

In the second embodiment, an example has been given in which the evaluation terminal 20 is a smartphone or a tablet terminal. The smartphone or the tablet terminal may include the presenter 14a and the input unit 16a, and the first signal processor 112a, the second signal processor 112b, the calculator 12a, the determiner 13a, and the storage unit 15a may be provided in a server connected through a communication network such as the Internet.

An example has been given in which the reference values and thresholds of the individual biological indices are stored in the storage unit provided in the evaluation terminal. Alternatively, the reference values and thresholds may be stored in a server on the Internet and may be transmitted to the evaluation terminal as necessary.

In an embodiment of the present disclosure, skin conductance is used as one of indices for determining a factor for stress. The index is not limited as long as the index indicates mental perspiration. For example, an index acquired by measuring a potential or current value of skin, such as skin resistance, may be used, or an index acquired by measuring the amount of moisture, such as humidity on a skin surface, may be used.

In the second embodiment, an example has been given in which a skin conductance or a skin temperature is measured at a palm. Alternatively, a skin conductance or a skin temperature may be measured at a part of the face or at the sole of a foot, where mental perspiration is likely to occur.

In an embodiment of the present disclosure, a mock job interview in a monitoring test is used as a specific example of an interpersonal-related factor, which is one of factors for stress. The present disclosure is not limited thereto. For example, an interpersonal-related factor may be a factor that causes a measurement subject to feel anxiety or nervous when being involved with a person, such as an interpersonal relationship in a workplace or a private life, speaking in public, or negotiation with somebody.

In an embodiment of the present disclosure, pain from electrical stimulations is used as a specific example of pain, which is one of factors for stress. The present disclosure is not limited thereto, and the pain may be any kind of pain causing fear or patience, for example, physical pain such as pain of a bruise, headache, toothache, or pain of a tear, or pain caused by physical stimulations such as scratching, stinging, cutting, or hitting.

In an embodiment of the present disclosure, mental arithmetic and voice paper-rock-scissors questions, which are tasks requiring thinking, are used as a specific example of tasks causing thinking-induced fatigue, which is one of factors for stress. The present disclosure is not limited thereto, and the thinking-induced fatigue may be any kind of fatigue induced by continuous thinking, for example, fatigue from working on a personal computer or fatigue from an intellectual activity, such as an experiment requiring concentration.

One embodiment of the present disclosure is useful as a stress evaluation device capable of easily and accurately determining a factor for stress of a measurement subject from a plurality of types of biological indices.

What is claimed is:

1. A stress evaluation device comprising:
a first sensor that measures a heart rate and a heart rate variability of a measurement subject;
a calculator that calculates (i) an amount of change in heart rate and (ii) an amount of change in heart rate variability; and
a determiner that determines a factor for stress of the measurement subject in accordance with (i) the amount of change in heart rate and (ii) the amount of change in heart rate variability and that outputs information based on a determination result, wherein
the amount of change in heart rate is an amount of change from a reference value that is an at-rest heart rate of the measurement subject to the heart rate measured by the first sensor,
the amount of change in heart rate variability is an amount of change from a reference value that is an at-rest heart rate variability of the measurement subject to the heart rate variability measured by the first sensor, and
the determiner makes
(I) a comparison between relative magnitudes of the amount of change in heart rate and a first threshold, and
(II) a comparison between relative magnitudes of the amount of change in heart rate variability and a second threshold
to determine the factor for the stress.

2. The stress evaluation device according to claim 1, wherein
the amount of change in heart rate is an amount of change to the heart rate measured at a first time,
the amount of change in heart rate variability is an amount of change to the heart rate variability measured at a second time,
the first threshold is the heart rate measured at a certain time different from the first time and the second time relative to the at-rest heart rate of the measurement subject, and
the second threshold is the heart rate variability measured at the certain time relative to the at-rest heart rate variability of the measurement subject.

3. The stress evaluation device according to claim 1, wherein in a case where the amount of change in heart rate is larger than the first threshold and the amount of change in heart rate variability is larger than the second threshold, the determiner determines that the factor for the stress is an interpersonal-related factor.

4. The stress evaluation device according to claim 1, wherein in a case where the amount of change in heart rate is larger than the first threshold and the amount of change in heart rate variability is smaller than the second threshold, the determiner determines that the factor for the stress is pain.

5. The stress evaluation device according to claim 1, wherein in a case where the amount of change in heart rate is smaller than the first threshold and the amount of change in heart rate variability is larger than the second threshold, the determiner determines that the factor for the stress is thinking-induced fatigue.

6. The stress evaluation device according to claim 1, wherein the determiner further determines an intensity of the stress in accordance with a difference between the amount of change in heart rate and the first threshold and a difference between the amount of change in heart rate variability and the second threshold, and outputs a determination result as the information based on the determination result.

7. The stress evaluation device according to claim 1, further comprising:
a second sensor that measures at least one of a skin conductance or a skin temperature of the measurement subject, wherein
the calculator further calculates (iii) an amount of change in skin conductance or an amount of change in skin temperature,
the amount of change in skin conductance is an amount of change from a reference value that is an at-rest skin conductance of the measurement subject to the skin conductance measured by the second sensor,
the amount of change in skin temperature is an amount of change from a reference value that is an at-rest skin temperature of the measurement subject to the skin temperature measured by the second sensor, and
the determiner makes, in addition to the (I) and the (II),
(III) a comparison between relative magnitudes of the amount of change in skin conductance or the amount of change in skin temperature and a third threshold
to determine the factor for the stress of the measurement subject, and outputs information based on a determination result.

8. The stress evaluation device according to claim 7, wherein
the amount of change in heart rate is an amount of change to the heart rate measured at a first time,
the amount of change in heart rate variability is an amount of change to the heart rate variability measured at a second time,
the amount of change in skin conductance or the amount of change in skin temperature is an amount of change to the skin conductance or the skin temperature measured at a third time,
the first threshold is the heart rate measured at a certain time different from the first time, the second time, and the third time relative to the at-rest heart rate of the measurement subject,
the second threshold is the heart rate variability measured at the certain time relative to the at-rest heart rate variability of the measurement subject, and
the third threshold is the skin conductance measured at the certain time relative to the at-rest skin conductance of the measurement subject or the skin temperature measured at the certain time relative to the at-rest skin temperature of the measurement subject.

9. The stress evaluation device according to claim 7, wherein in a case where the amount of change in heart rate is larger than the first threshold, the amount of change in heart rate variability is larger than the second threshold, and the amount of change in skin conductance or the amount of change in skin temperature is larger than the third threshold, the determiner determines that the factor for the stress is an interpersonal-related factor.

10. The stress evaluation device according to claim 7, wherein in a case where the amount of change in heart rate is larger than the first threshold, the amount of change in heart rate variability is smaller than the second threshold, and the amount of change in skin conductance or the amount of change in skin temperature is larger than the third threshold, the determiner determines that the factor for the stress is pain.

11. The stress evaluation device according to claim 7, wherein in a case where the amount of change in heart rate is smaller than the first threshold, the amount of change in heart rate variability is larger than the second threshold, and the amount of change in skin conductance or the amount of change in skin temperature is smaller than the third threshold, the determiner determines that the factor for the stress is thinking-induced fatigue.

12. The stress evaluation device according to claim 7, wherein the determiner further determines an intensity of the stress in accordance with a difference between the amount of change in heart rate and the first threshold, a difference between the amount of change in heart rate variability and the second threshold, and a difference between the amount of change in skin conductance or the amount of change in skin temperature and the third threshold, and outputs a determination result as the information based on the determination result.

13. The stress evaluation device according to claim 1, wherein the heart rate variability is obtained by performing frequency analysis on heartbeat intervals of the measurement subject.

14. The stress evaluation device according to claim 1, further comprising:
a presenter that presents the information based on the determination result output by the determiner, wherein
the information includes at least one selected from the group consisting of the factor for the stress, an intensity of the stress, and measures for reducing the stress.

15. The stress evaluation device according to claim 14, wherein the presenter presents the information by using a sound.

16. The stress evaluation device according to claim 14, wherein the presenter presents the information by using an image.

17. A stress evaluation method comprising:
acquiring a measured heart rate and a measured heart rate variability of a measurement subject;
calculating (i) an amount of change in heart rate and (ii) an amount of change in heart rate variability; and
determining a factor for stress of the measurement subject in accordance with (i) the amount of change in heart rate and (ii) the amount of change in heart rate variability and outputting information based on a determination result, wherein
the amount of change in heart rate is an amount of change from a reference value that is an at-rest heart rate of the measurement subject to the measured heart rate,
the amount of change in heart rate variability is an amount of change from a reference value that is an at-rest heart rate variability of the measurement subject to the measured heart rate variability, and
the determining includes making
(I) a comparison between relative magnitudes of the amount of change in heart rate and a first threshold, and
(II) a comparison between relative magnitudes of the amount of change in heart rate variability and a second threshold
to determine the factor for the stress.

18. The stress evaluation method according to claim 17, wherein
the acquiring includes acquiring at least one of a measured skin conductance or a measured skin temperature of the measurement subject,
the calculating includes calculating (iii) an amount of change in skin conductance or an amount of change in skin temperature,
the amount of change in skin conductance is an amount of change from a reference value that is an at-rest skin conductance of the measurement subject to the measured skin conductance,
the amount of change in skin temperature is an amount of change from a reference value that is an at-rest skin temperature of the measurement subject to the measured skin temperature, and
the determining includes making, in addition to the (I) and the (II),
(III) a comparison between relative magnitudes of the amount of change in skin conductance or the amount of change in skin temperature and a third threshold
to determine the factor for the stress of the measurement subject, and outputting information based on a determination result.

19. A non-transitory computer-readable medium having a program stored thereon, the program causing a computer to execute the stress evaluation method according to claim 17.
